SEQUENCE LISTING (5 sequences)

SEQ ID NO: 1 - DNA, 25 bp, artificial sequence
  Primer for cloning of His6 tagged human rpL35
catgccatggccaagatcaaggctc

SEQ ID NO: 2 - DNA, 25 bp, artificial sequence
  Primer for cloning His6 tagged human rpL35
ctctagattcagtcagatctcagtg

SEQ ID NO: 3 - protein, 123 aa, Homo sapiens [sequence omitted]

SEQ ID NO: 4 - protein, 120 aa, Saccharomyces cerevisiae
MAGVKAYELRTKSKEQLASQLVDLKKELAELKVQKLSRPSLPKIKTVRKSIACVLTVINEQQREAVRQLYKGKKYQPKDLRAKKTRALRRALTKFEASQVTEKQRKKQIAFPQRKYAIKA

SEQ ID NO: 5 - protein, 136 aa, artificial sequence
  His-tagged human rpL35 protein
GAMAKIKARDLRGKKKEELLKQLDDLKVELSQLRVAKVTGGAASKLSKIRVVRKSIARVLTVINQTQKENLRKFYKGKKYKPLDLRPKKTRAMRRRLNKHEENLKTKKQQRKERLYPLRKYAVKAAAALEHHHHHH

(19) United States
(12) Patent Application Publication
(10) Pub. No.: US 2023/0288402 A1
(43) Pub. Date: Sep. 14, 2023

(54) MOLECULES TARGETING RIBOSOMAL PROTEIN RPL35/UL29 FOR USE IN THE TREATMENT OF DISEASES, IN PARTICULAR EPIDERMOLYSIS BULLOSA (EB)

(71) Applicant: KBHB CONSULT GMBH, SALZBURG (AT)

(72) Inventors: HANNELORE BREITENBACH-KOLLER, SALZBURG (AT); JÖRG VON HAGEN, PFUNGSTADT (DE); HELMUT HINTNER, SALZBURG (AT); FRIEDRICH LOTTSPEICH, STOCKDORF (DE); HANS-WERNER MEWES, SEEFELD (DE); NORBERT MÜLLER, SEEFELD (DE); ANDREAS FRIEDRICH, BERCHTESGADEN (DE); KATHARINA ELSENSOHN, ELSBETHEN (AT); CLAUDIA MOSSHAMMER, MITTERSILL (AT); MICHAEL WIESSNER, SALZBURG (AT); THOMAS KARL, PUCH BEI HALLEIN (AT); JAN SCHERNTHANER, SALZBURG (AT); ADRIANA RATHNER, LINZ (AT)

(21) Appl. No.: 18/018,908

(22) PCT Filed: Jul. 30, 2021
(86) PCT No.: PCT/EP2021/071289

§ 371 Date: Jan. 31, 2023

(30) Foreign Application Priority Data
Jul. 31, 2020 (DE) 10 2020 120 218.0

(51) Int. Cl. G01N 33/50

(52) U.S. Cl. CPC G01N 33/5023; G01N 2500/10

(57) ABSTRACT

The present invention relates to a method for identifying a pharmaceutically active compound that modulates the rpL35 (rpL35/rpL29)-dependent translation of at least one mRNA in a mammalian cell. The mRNA may comprise a premature termination codon (PTC), undergoes premature translation termination, causes programmed −1 ribosomal frame shifting (−1PRF), or is a polycistronic mRNA. Furthermore a respective screening system, methods of treating or preventing a disease or condition, and compounds that modulate the rpL35 (rpL35/rpL29)-dependent translation, in particular atazanavir or derivatives thereof and artemisinin or artesunate or derivatives thereof are provided.

The present invention relates to a method for identifying a pharmaceutically active compound that modulates the rpL35 (rpL35/rpL29)-dependent translation of at least one mRNA in a mammalian cell. The mRNA may comprise a premature termination codon (PTC), undergoes premature translation termination, causes programmed −1 ribosomal frameshifting (−1PRF), or is a polycistronic mRNA. Furthermore a respective screening system, methods of treating or preventing a disease or condition, and compounds that modulate the rpL35 (rpL35/rpL29)-dependent translation, in particular atazanavir or derivatives thereof and artemisinin or artesunate or derivatives thereof are provided.

### BACKGROUND OF THE INVENTION

There is no cure for most of the currently known genetic diseases. Genetic diseases are generally those in which a change (e.g. mutation) is inherited in a particular gene or has developed in the germ line. This mutation then produces or results in a specific clinical manifestation of a disease. Out of the approximately 10,000 rare diseases (also called orphan diseases, occur in less than 0.01% of a given population; In total, all rare diseases together affect up to 10% of the population, i.e. about 500 million people worldwide), approximately 6,000 are genetic diseases involving a mutation as mentioned above.

There are more than 1800 distinctly inherited human genetic disorders where nonsense mutations (in-frame, single-point alterations in the genetic code that prematurely stop the translation process of proteins producing non-functional, shortened molecules) cause disease in an appreciable percentage of patients. In-frame premature termination codons (PTCs) account for about 11% of all described gene defects causing human genetic diseases, including rare diseases, and are often associated with a severe phenotype. The specific genetic mutational event of a PTC mutation during translation of the PTC-affected mRNA leads to premature termination of protein synthesis. A PTC mutation replaces an mRNA sense codon with an unscheduled stop codon/nonsense codon, a signal for termination of protein synthesis. This produces a truncated, potentially non-functional and even harmful protein.

Despite this, there is a basal cellular rescue mechanism to still generate a full length and functional protein from a PTC-affected mRNA, the so-called “read through” that involves the use of a near-cognate tRNA which is able to interpret the PTC as a sense codon in order to still incorporate an amino acid into the growing protein chain. The amino acid that is inserted by the read-through may be identical to the original amino acid of the wild type protein or not. Nevertheless, there is only a very small selected subset of amino acids that can be substituted, and for which no drastic effect on protein structure and function has been reported. Furthermore, the basal read through level is low, and—depending on the sequence context of the PTC—read through may vary between 1 in 10.000 mRNA translation events to 1 in 100 events. Therefore, basal read through levels do not provide enough full length protein in order to avoid/overcome the manifestation of a disease phenotype, in particular orphan disease phenotypes.

U.S. Pat. No. 7,927,791 relates to a method for screening and identifying compounds that modulate premature translation termination and/or nonsense-mediated messenger ribonucleic acid (“mRNA”) by interacting with a preselected target ribonucleic acid (“RNA”). In particular, the present invention relates to identifying compounds that bind to regions of the 28S ribosomal RNA (“rRNA”) and analogs thereof.

WO 2012/142542 relates to methods to identify molecules that binds in the neomycin binding pocket of a bacterial ribosome using structures of an intact bacterial ribosome that reveal how the ribosome binds tRNA in two functionally distinct states

Dabrowski et al. (in: Advances in therapeutic use of a drug-stimulated translational readthrough of premature termination codons; Mol Med. 2018; 24: 25) disclose translational read through of PTCs induced by pharmaceutical compounds as a promising way of restoring functional protein expression and reducing disease symptoms, without affecting the genome or transcriptome of the patient. While in some cases proven effective, the clinical use of readthrough-inducing compounds would still be associated with many risks and difficulties. The article focuses on problems directly associated with compounds used to stimulate PTC readthrough, such as their interactions with the cell and organism, their toxicity and bioavailability (cell permeability; tissue deposition etc.). Various strategies designed to overcome these problems are discussed.

Keeling K M, et al. (in: Therapeutics based on stop codon readthrough. Annu Rev Genomics Hum Genet. 2014; 15:371-394. doi:10.1146/annurev-genom-091212-153527) disclose that nonsense suppression therapy encompasses approaches aimed at suppressing translation termination at in-frame premature termination codons (PTCs, also known as nonsense mutations) to restore deficient protein function. They examine the current status of PTC suppression as a therapy for genetic diseases caused by nonsense mutations and discuss the mechanism of PTC suppression as well as therapeutic approaches under development to suppress PTCs. The approaches considered include readthrough drugs. Finally, they consider how PTC suppression may play a role in the clinical treatment of genetic diseases caused by nonsense mutations.

The problem of all therapeutic approaches for rare and more common genetic disorders, results from the fact that although the genes involved and their mutations are known exactly, often the response to therapeutic interventions in the cellular and molecular network can only be poorly predicted, if at all. Also there is insufficient functional characterization of the therapeutic compounds and their effects on cellular targets and metabolic pathways. This is exemplified by the development of synthetically developed drugs with complex and combinatorially generated structures, where toxic effects on cellular components (off targets) become evident only in later phases of clinical trials, and such product candidates ultimately fail because of these unwanted side effects.

At present, therapeutic interventions to increase readthrough comprise aminoglycoside antibiotics, derivatives thereof and synthetically developed drugs that either have severe side effects and cannot be administered continuously or do not promote increased read through in all patients.

As a prototypical orphan disease with a PTC defect, the inventors studied severe junctional Epidermolysis bullosa (EB, gs-JEB), a hitherto incurable and in most cases fatal blistering skin disorder. gs-JEB is caused by loss of function mutations in the genes LAMA3, LAMB3 or LAMC2, which in each case lead to complete loss of the trimeric laminin 332 complex, composed of the proteins laminin α3 (Lama3), laminin β3 (Lamb3) and laminin γ2 (Lamc2). Without the Lamb332 complex, no functional connection between epidermis and dermis can be established. Patients suffer from extreme blistering of the skin and mucous membranes, of the digestive tract, chronic infections, and purulent wounds and drastically reduced wound healing.

No approved targeted systemic therapy is available to PTC mutations in EB, in particular not for gs-JEB. Therapeutic options are limited to palliative care. Individual therapeutic strategies such as protein replacement therapy and bone marrow stem cell transplantation have been successful in cell culture or have directly, at least partially, cured individual patients by transplanting gene-corrected, patient-derived keratinocyte epithelia (Hirsch T, Rothoeft T, Teig N, et al. Regeneration of the entire human epidermis using transgenic stem cells. Nature. 2017; 551(7680):327-332. doi:10.1038/nature24487).

Nguyen, H. L., et al. (in: Erythromycin leads to differential protein expression through differences in electrostatic and dispersion interactions with nascent proteins. Sci Rep 8, 6460 (2018). https://doi.org/10.1038/s41598-018-24344-9) examine interactions of the macrolide erythromycin with the ribosome.

Kwong A. et al. (in: Gentamicin Induces Laminin 332 and Improves Wound Healing in Junctional Epidermolysis Bullosa Patients with Nonsense Mutations. Mol Ther. 2020 May 6; 28(5):1327-1338. doi: 10.1016/j.ymthe.2020.03.006) disclose that using primary keratinocytes from three GS-JEB patients, gentamicin induced functional laminin 332 that reversed a JEB-associated, abnormal cell phenotype.

Consequently, development of alternative therapeutic interventions is in high demand. It is therefore an object of the present invention, to provide new strategies and interventions for the therapy of diseases and conditions of diseases and conditions related to or caused by PTC-affected mRNAs. Other objects and advantages of the present invention will become apparent to the person of skill when studying the following more detailed description of the present invention, including the Figures and examples.

One way to overcome the above obstacles could be to employ already approved synthetic drugs as re-purposed drugs for diseases other than their original indication/application (off-label). Furthermore, natural (naturally derived) drugs and products show an enormous structural and chemical diversity, which can not be achieved by any synthetic drug library, since about half of all chemical structures that are described for natural products simply do not from part of synthetic drug libraries. In the biological context, in many instances natural products are already evolutionarily optimized as drug-like molecules because they are used by microorganisms, plants or animals as a chemical intervention against competing organisms or pathogens. Currently, less than 10% of all microorganisms are examined for their spectrum of possible therapeutic compounds. This tremendous reservoir of potentially therapeutically active natural products is thus available as a untapped resource for high-potency drug discovery while potentially minimizing side effects in more common diseases, and in particular in orphan diseases, such as EB.

In the majority of gs-JEB cases, the LAMB3 gene is affected (about 80%). Until today, about 90 different mutations are described, of which almost all are PTC mutations. A recent study on 65 gs-JEB patients (with both genders within the patient cohort) showed that the R635X-PTC mutation is present in 84% of patients with a mutated LAMB3 gene. Therefore, this mutation is a primary therapeutic target among the LAMB3 mutations to develop therapies that suppress this PTC mutation. One such therapeutic approach is the use of aminoglycoside antibiotics, such as gentamicin, and their derivatives, which enhance the rare, basal, endogenous process of PTC reading, thereby increasing the production of a full-length protein. This has been demonstrated in patients with PTC mutation in the gene for cystic fibrosis, CFTR (Wilschanski M, Yahav Y, Yaacov Y, et al. Gentamicin-induced correction of CFTR function in patients with cystic fibrosis and CFTR stop mutations. N Engl J Med. 2003; 349(15):1433-1441. doi:10.1056/NEJMoa022170), but also in cell culture studies for the DMD gene in muscular dystrophy (Bidou L, Hatin I, Perez N, Allamand V, Panthier J J, Rousset J P. Premature stop codons involved in muscular dystrophies show a broad spectrum of readthrough efficiencies in response to gentamicin treatment. Gene Ther. 2004; 11(7):619-627. doi:10.1038/sj.gt.3302211), for the ATM gene in Ataxia teleangiectatica (Lai et al., 2004) and for the APC gene in colon carcinoma (Zilberberg A, Lahav L, Rosin-Arbesfeld R. Restoration of APC gene function in colorectal cancer cells by aminoglycoside- and macrolide-induced read-through of premature termination codons. Gut. 2010; 59(4):496-507. doi:10.1136/gut.2008.169805). In cell culture, it was recently shown that readthrough enhancement of Lamb3 protein production with the R635XPTC mutation is possible. However, this is achieved only at a very high, therapeutically not applicable dose of gentamicin, which delivers 30% of the normal level of Lamb3 protein expression. Even at lower and therapeutically useful doses in the treatment of other diseases with PTC mutations, the clinical use of aminoglycosides is limited because of severe side effects (nephrotoxicity and ototoxicity), so that also other therapeutic approaches must be sought (Wilschanski et al., 2003).

Bauer et al. (in: Specialized yeast ribosomes: a customized tool for selective mRNA translation. PLoS One. 2013; 8(7):e67609. doi:10.1371/journal.pone.0067609) describe diploid yeast strains, each deficient in one or other copy of the set of ribosomal protein (RP) genes, to generate eukaryotic cells carrying distinct populations of altered ‘specialized’ ribosomes. Using the strains, a screen identified specialized ribosomes with reduced levels of RP L35B as showing enhanced synthesis of full-length LAMB3 in cells expressing a LAMB3-PTC mutant. It was speculated that the rational modification of a eukaryotic ribosome could customize increased translation of a specific, disease-associated mRNA and may represent a novel therapeutic strategy for the future.

As an efficient systemic therapy with low-side-effects is not available for diseases related to undesirable translation products related to a mammalian ribosomal protein rpL35, such as for example Epidermolysis bullosa caused by PTC mutations in the LAMB3, collagen VII or collagen XVII genes, as well as for those cancers with PTC mutations manifesting in later stages of the disease, such as PTC mutations in the genes for p53 and APOBEC, is not available, new therapeutic approaches are required. It is therefore an object of the present invention to provide such new approaches, methods and means. Other objects and advantages will become apparent to the person of skill when studying the description of the present invention.

In a first aspect of the present invention, the above object is solved by a method for identifying a pharmaceutically active compound that modulates the rpL35 (rpL35/rpL29)-dependent translation of at least one mRNA in a mammalian cell, comprising a) contacting rpL35 or a functional fragment thereof with at least one candidate compound in the presence of said at least one mRNA to be translated, and
b) detecting the modulation of the translation of said at least one mRNA compared to the translation in the absence of said at least one candidate compound, wherein a modulation of the translation of said at least one mRNA is indicative for said pharmaceutically active compound.

Preferably, said at least one mRNA comprises a premature termination codon (PTC), undergoes premature translation termination, causes programmed −1 ribosomal frameshifting (−1PRF), or is a polycistronic mRNA. More preferably, said method furthermore comprises detecting a binding of said at least one candidate compound to a fragment of rpL35, wherein said fragment comprises from about 70 to about 100 of the N-terminal amino acids of the mammalian rpL35, or said detecting of binding to rpL35 or the fragment thereof is performed as a pre-screening before contacting said at least one candidate compound with said rpL35.

In a second aspect of the present invention, the above object is solved by a providing a screening system for identifying a pharmaceutically active compound that modulates the rpL35 (rpL35/rpL29)-dependent translation of at least one mRNA in a mammalian cell, comprising a eukaryotic cell recombinantly expressing a mammalian rpL35 or a fragment of a mammalian rpL35, wherein said fragment comprises from about 70 to about 100 of the N-terminal amino acids of rpL35, for example according to SEQ ID NO: 3, an expression construct for recombinantly expressing at least one mRNA to be tested, and, optionally, one or more candidate compounds to be tested.

In a third aspect of the present invention, the above object is solved by providing a compound that modulates the rpL35 (rpL35/rpL29)-dependent translation of at least one mRNA in a mammalian cell for use in the prevention or treatment of diseases or condition caused by i) an mRNA comprising a premature termination codon (PTC), ii) an mRNA that undergoes premature translation termination, iii) programmed −1 ribosomal frameshifting (−1PRF), or iv) the expression of a polycistronic mRNA. Preferably, said disease or condition is selected from Epidermolysis bullosa, and viral infections, in particular retroviral infections, such as HIV-1 or coronavirus, for example SARS CoV2.

In a fourth aspect of the present invention, the above object is solved by a method of modulating the rpL35 (rpL35/rpL29)-dependent translation of at least one mRNA in a mammalian cell, comprising contacting said cell with an effective amount of atazanavir or derivatives thereof and artemisinin or artesunate or derivatives thereof, or combinations thereof.

In a fifth aspect of the present invention, the above object is solved by a method of treating or preventing a disease or condition caused by i) an mRNA comprising a premature termination codon (PTC), ii) an mRNA that undergoes premature translation termination, iii) programmed −1 ribosomal frameshifting (−1PRF), or iv) the expression of a polycistronic mRNA in a mammalian cell, comprising providing an effective amount of at least one compound that modulates the rpL35 (rpL35/rpL29)-dependent translation of said mRNS according to any of i) to iv) to a patient or subject in need of said treatment or prevention.

The present invention is based on the surprising finding that human ribosomal protein rpL35 (rpL35/rpL29) can be used as a target for a tailor-made modulation of the translation of certain mRNAs into proteins. At present, there is no technology that by systemic modification of a ribosomal protein customizes the ribosome for increase or decrease in protein production of a given protein. Boosting or reduction of protein species is desirable in medical applications, in biotechnological applications and in cosmetic and anti-aging interventions.

Ultimately, the present invention is in the field of specialized ribosomes, and targeting ribosomal proteins (RP) offers new routes for the treatment of severe inherited diseases such as EB (see also: Dalla Venezia N., et al. Emerging Role of Eukaryote Ribosomes in Translational Control. Int J Mol Sci. 2019; 20(5):1226. Published 2019 Mar. 11. doi:10.3390/ijms20051226; Ferretti M B, Karbstein K. Does functional specialization of ribosomes really exist?. RNA. 2019; 25(5):521-538. doi:10.1261/rna.069823.118). Recent results have underscored the importance of translational control in regulation of gene expression, augmenting the traditional role of transcription. Mis-regulation of translation is a leading cause of many diseases. Viruses use a variety of mechanisms to co-opt the translational machinery to facilitate their replication, including manipulation of translation initiation factors, and specific RNA structures to guide translation within their genomes. Translational control has been implicated in human cancer, with changes in protein synthesis caused by up-regulation or changed functions of initiation factors. Finally, many genetic diseases disrupt translation through premature termination codons (Chen J, et al. The molecular choreography of protein synthesis: translational control, regulation, and pathways. Q Rev Biophys. 2016; 49:e11. doi:10.1017/S0033583516000056). This invention provides progress towards this concept in order to harness the potential of targeting translation therapeutically.

EP2251437 discloses a two-step specialized ribosome screen (2SSRC) which is able to screen for ribosomal protein targets specifically regulating protein synthesis of a protein of interest.

These screens provide a direct readout of protein synthesis. The assay used in the screen can be employed for all follow up steps including pre-clinical studies, or testing a small molecule binder which targets the ribosomal protein for customizing protein expression. In yeast and human cells, subpopulations of cytoplasmic ribosomes can be generated, by providing altered functional availability of individual ribosomal proteins. Such heterologous or specialized ribosomes are tailored to increase or decrease protein expression of selected mRNAs, while leaving bulk protein expression unaltered.

In a first aspect of the present invention, a method for identifying a pharmaceutically active compound that modulates the rpL35 (rpL35/rpL29)-dependent translation of at least one mRNA in a mammalian cell (“modulator”) is provided. The method comprises the steps of contacting rpL35 or a functional fragment thereof with at least one candidate compound in the presence of said at least one mRNA to be translated, and detecting the modulation of the translation of said at least one mRNA compared to the translation in the absence of said at least one candidate compound. A modulation of the translation of said at least one mRNA as detected is then indicative for said pharmaceutically active compound.

The term “contacting” in the present invention means any interaction between the potential modulator with the ribosomal protein or fragment thereof as described herein and/or a recombinant cell expressing said ribosomal protein or fragment thereof, whereby any of the two components can be independently of each other in a liquid phase, for example in solution, or in suspension or can be bound to a solid phase, for example, in the form of an essentially planar surface or in the form of particles, pearls or the like. In a preferred embodiment, a multitude of different potentially binding candidate compound are immobilized on a solid surface like, for example, on a compound library chip, and the ribosomal protein or fragment thereof as described herein is subsequently contacted with such a chip.

The method according to the present invention seeks to a pharmaceutically active compound that modulates the rpL35 (rpL35/rpL29)-dependent translation of at least one mRNA in a mammalian cell (“modulator”), preferably in a human cell. Conveniently, the format of the method can be quite flexible, the method requires a suitable combination of the three components rpL35 (either isolated or in combination with other ribosomal components) or a functional fragment thereof, the at least one mRNA to be translated, and the at least one pharmaceutically active candidate compound, i.e. the substance that shall be screened/identified for the activity to modulate the rpL35 (rpL35/rpL29)-dependent translation of at least one mRNA in a mammalian cell. This combination can be provided as a cellular system (i.e. functioning in a cell, like in a yeast or mammalian cell culture), and the components can be provided recombinantly in part or fully, or the system can be in vitro, for example as an in vitro translation system that can be readily adjusted for the purposes of the present invention, if required. Examples are Hela cell in vitro translation assays and human HaCat PTC/PTC model cells.

Preferred is the method according to the present invention, which includes the pre-identification of the translation of the at least one mRNA as rpL35 (rpL35/rpL29)-dependent, preferably to at least in part, more preferably to a substantial part thereof. A preferred tool for identifying such dependency uses the two-step specialized ribo-screen (2SSRS) as disclosed by, e.g. EP2251437, herewith incorporated by reference, which identifies target ribosomal proteins that tailor protein synthesis of mRNAs of proteins of interest (POIs). Comparative protein synthesis assays also identify mRNAs that are preferentially translated by distinct populations of specialized ribosomes. Furthermore, proteomic analysis can identify the absolute protein concentration in a given sample.

rpL35 (either isolated or in combination with other ribosomal components) or a functional fragment thereof, as described herein, is then contacted (see above) with said at least one candidate compound in the presence of said at least one mRNA to be translated. Then, the modulation of the translation of said at least one mRNA compared to the translation in the absence of said at least one candidate compound is detected. Translation can be detected directly, for example by detecting the amount and/or presence and/or size (length) of the polypeptide as produced. This can be achieved with mass spectrometry, NMR, ELISA, labels that are include into the polypeptide, like labelled amino acids, luminescence constructs (renilla and/or firefly), fusions (GFP), and the like. Preferably, the detection involves a quantification of the translation product, and preferred is the method according to the present invention, wherein said modulation leads to or produces an increase or decrease of said rpL35 (rpL35/rpL29)-dependent translation of said at least one mRNA. Specific examples of modulation would be an increase of the translation of a polypeptide as produced by readthrough over PTC codons, an inhibition (reduction) of polypeptides translated after programmed −1 ribosomal frameshifting, or the amount or ration (to each other) of polypeptides translated from a polycistronic mRNA.

Detecting the modulation of the translation of said at least one mRNA in case of mRNAs comprising a premature termination codon (PTC), mRNAs undergoing premature translation termination, mRNAs causing programmed −1 ribosomal frameshifting (−1PRF), or a polycistronic mRNA preferably includes a detecting of whether a certain full-length or “correct” polypeptide has been made (like in the context of PTC), and/or whether a set of polypeptides has been made or not (like in the context of viral polycistronic mRNAs), and whether these translation products have been modulated in their sizes, amounts, and/or composition, as the case may require.

In support of the method according to the invention, two small molecule binders and prospective modulators of rpL35 translation have been identified by a combination of bioinformatic studies, molecular docking studies, and in vitro NMR studies. The first molecule identified is artesunate (formula 1),

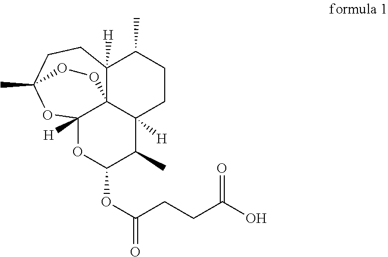 [SMILES: C[C@H]1[C@H](OC(=O)CCC(=O)O)O[C@@H]2O[C@]3(C)CC[C@H]4[C@H](C)CC[C@@H]1[C@@]24OO3]

a derivative of artemisinin (formula II),

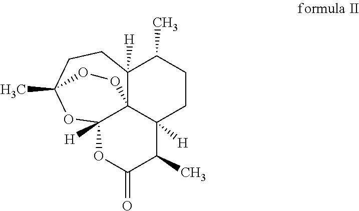

a sesquiterpene lactone containing an unusual peroxide bridge. The endoperoxide 1,2,4-trioxane ring is responsible for the drug's mechanism of action, in particular when used for the treatment of malarial and parasitic worm (helminth) infections. Artemisinin was shown to bind to a large number of targets suggesting that it acts in a promiscuous manner (Wang J, Zhang C J, Chia W N, et al. Haem-activated promiscuous targeting of artemisinin in Plasmodium falciparum. Nat Commun. 2015; 6:10111. doi:10.1038/ncomms10111). Interestingly, the publication of Wang et al.—among a large list of other protein targets—speculates about a binding to (in descending order with respect to confidence) 40S ribosomal protein S3, 60S ribosomal protein L4 (RPL4), 60S ribosomal protein L3 (RPL3), 40S ribosomal protein S19 (RPS19), 60S ribosomal protein L2 (RPL2), 60S ribosomal protein L27 (RPL27), 40S ribosomal protein S5, 40S ribosomal protein S21 (RPS21), 60S ribosomal protein L21 (RPL21), and ubiquitin-60S ribosomal protein L40. It is expected that derivatives and pharmaceutically acceptable salts of artemisinin and artesunate (see below) will show even improved properties to modulate rpL35 translation.

Artemisinin is presently also used for the treatment of malaria. WO 2010/012687A2 discloses formulations derived from Artemisia annua and their use in cosmetics and medicine. WO 2011/030223A2 discloses a process for the production of (2R)-dihydroartemisinic acid or (2R)-dihydroartemisinic acid esters from artemisinic acid or artemisinic acid esters, respectively. WO 2010/149215 relates to a pharmaceutical composition, in powder form, comprising artesunate as an anti-malarial agent.

Njuguna et al. (in: Artemisinin derivatives: a patent review (2006—present) Expert Opinion on Therapeutic Patents, Volume 22, 2012—Issue 10, Pages 1179-1203) provide a summary of patents published globally covering promising artemisinin derivatives and artemisinin-based drug combinations developed for use in various therapeutic areas.

The binding site of artesunate was characterized by NMR studies.

The second molecule identified is atazanavir (formula 3)

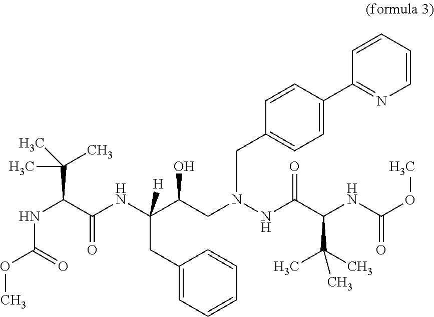 [SMILES: COC(=O)N[C@H](C(=O)N[C@@H](Cc1ccccc1)[C@@H](O)CN(Cc1ccc(-c2ccccn2)cc1)NC(=O)[C@@H](NC(=O)OC)C(C)(C)C)C(C)(C)C]

a synthetic tripeptide inhibitor of HIV protease I and II. This compound is used in treatment of HIV-infections, usually in combination with compound ritonavir, or coronavirus, like SARS CoV2. It is expected that derivatives and pharmaceutically acceptable salts of atazanavir (see below) will show even improved properties to modulate rpL35 translation.

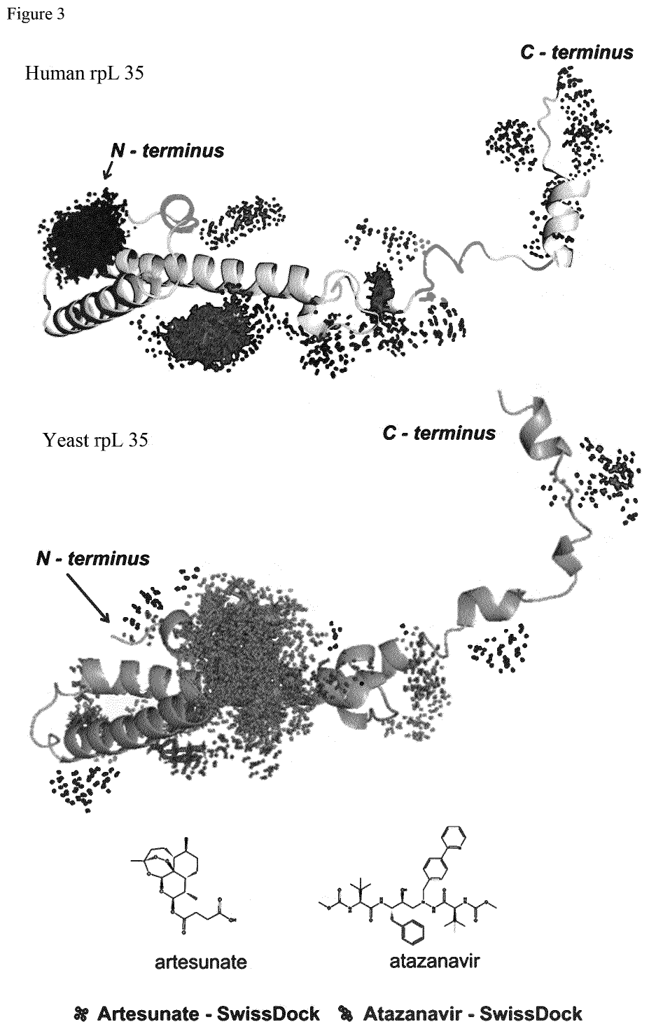

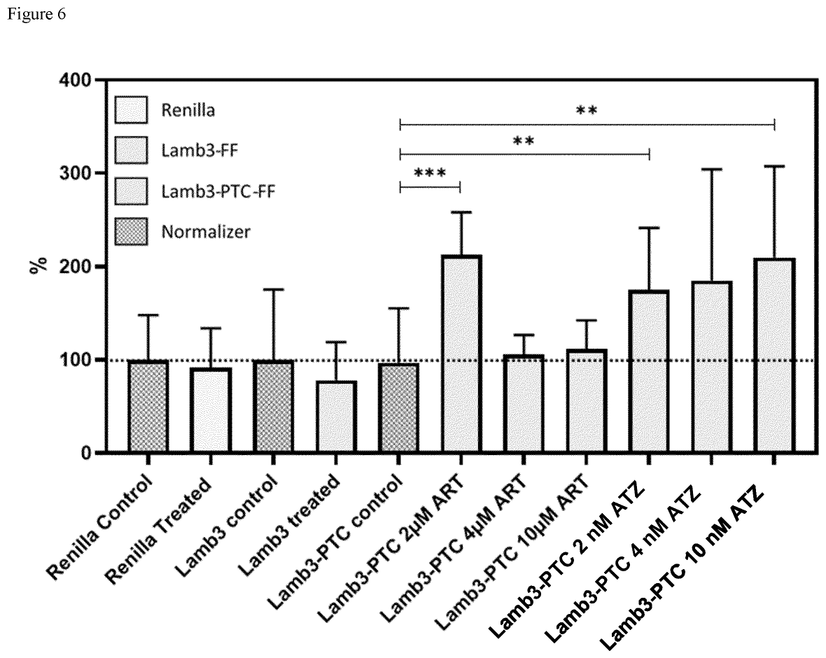

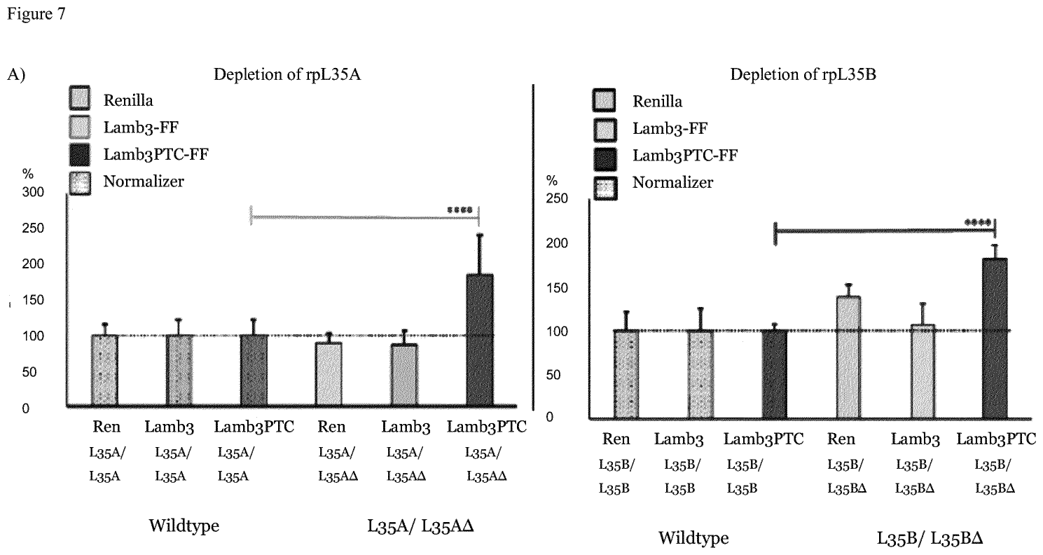

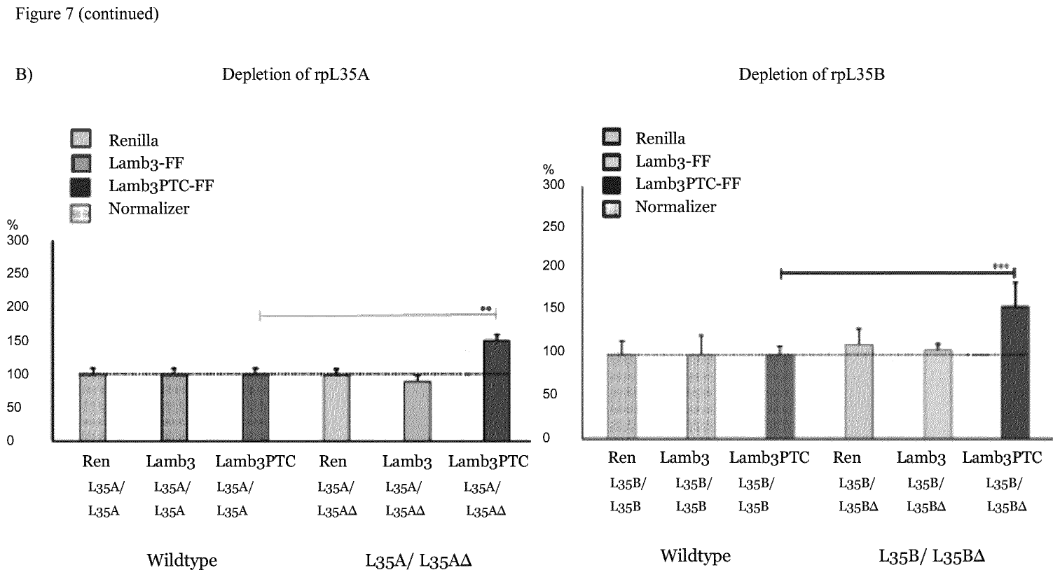

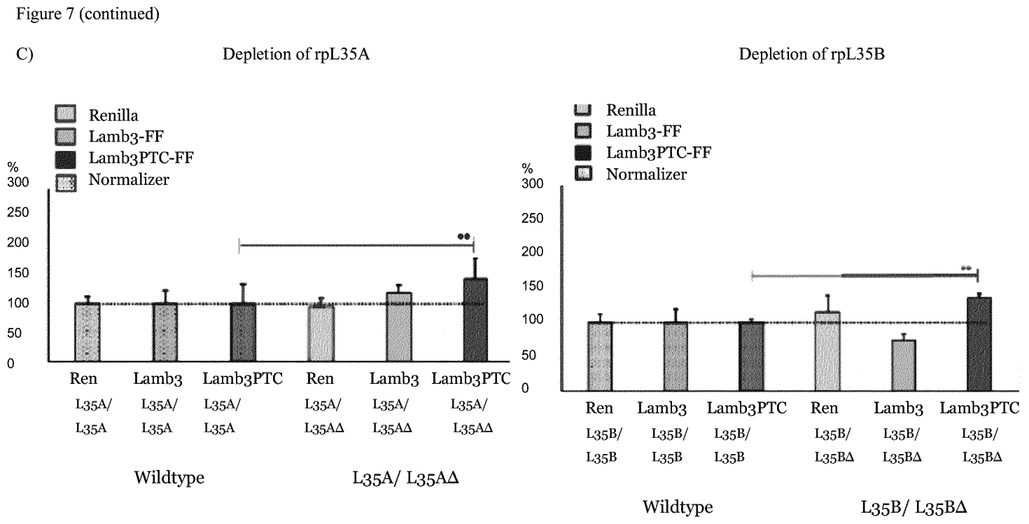

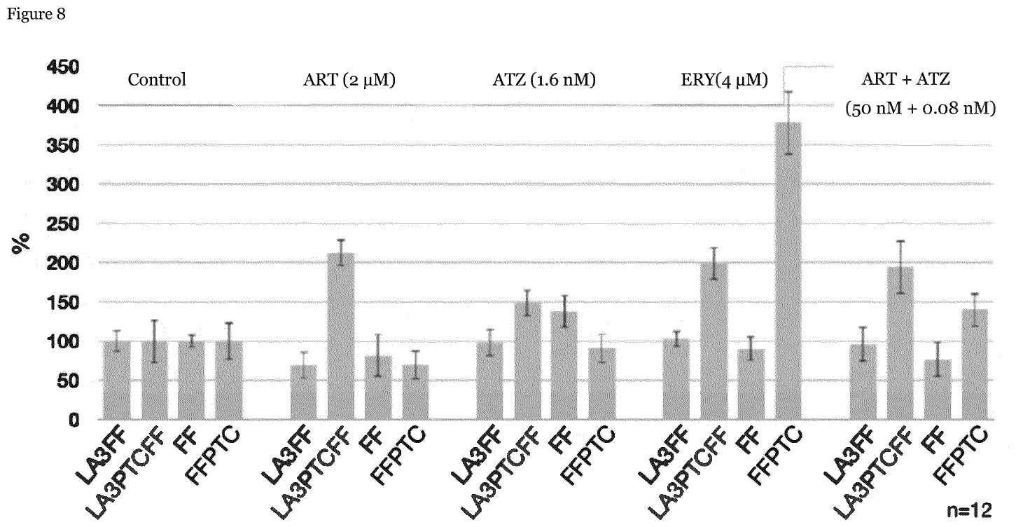

The inventors found that artesunate and atazanavir, respectively, bind to human rpL35 (FIG. 3). The inventors further found that administration of both artesunate and atazanavir increase Lamb3PTC expression in yeast cells (FIG. 6) in Hela in vitro translation assays (FIG. 7) and as first data indicate also prospectively in human HaCat PTC/PTC model cells. The inventors further identified that the use of artesunate and atazanavir in combination even further improves, and in particular synergistically improves, the effect compared to either drug delivered alone (see FIG. 8).

Here, the inventors disclose human ribosomal protein rpL35/uL29 as a target for protein therapy to at least partially overcome and “repair” a PTC gene defect in the rare disease Epidermolysis bullosa. In particular, the inventors found that human rpL35/uL29 is able to serve as cellular target for a drug action so that a functional protein is produced from an initial DNA that comprises a nonsense mutation (leading to a premature termination codon, PTC), here in the skin anchor protein LAMB3. The inventors show that in this way a treatment is achieved for the disorder, which is associated with the PTC mutation in the LAMB3 gene.

Similar to the mRNA comprising a premature termination codon (PTC), the present invention can overcome and “repair” the translation of an mRNA that undergoes premature translation termination, a programmed −1 ribosomal frameshifting (−1PRF), or overcome or “repair” (reduce or inhibit) the expression of a set of proteins derived from a polycistronic mRNA.

This will help to treat and alleviate and/or prevent respective disease that are related to these mRNA molecules, such as, for example, Epidermolysis bullosa and other PTC diseases, and viral infections, in particular retroviral infections, such as HIV-1 or HCV or coronavirus, for example SARS CoV2.

Examples for the interaction of ribosomal proteins with viral protein translation can be found in the literature. Li (in: Regulation of Ribosomal Proteins on Viral Infection. Cells. 2019; 8(5):508. Published 2019 May 27. doi:10.3390/cells8050508) discloses that ribosomal proteins could provide a new platform for antiviral therapy development, however, at present, antiviral therapeutics with ribosomal proteins involving in virus infection as targets is limited, and exploring antiviral strategy based on ribosomal proteins. Green et al. (in: Large Ribosomal Protein 4 Increases Efficiency of Viral Recoding Sequences” Journal of Virology 86 (2012): 8949-8958) disclose the effects of a host protein, large ribosomal protein 4 (rpL4), on the efficiency of viral recoding. Expression of rpL4 increases recoding of reporters containing retroviral readthrough and frameshift sequences, as well as the Sindbis virus leaky termination signal. Green L and Goff S P (in: Translational readthrough-promoting drugs enhance pseudoknot-mediated suppression of the stop codon at the Moloney murine leukemia virus gag-pol junction. J. Gen. Virol. 2015; 96(11):3411-3421. doi:10.1099/jgv.0.000284) then disclose the effects of readthrough-promoting drugs—aminoglycoside antibiotics and the small molecule ataluren—on the efficiency of readthrough of the stop codon in the context of the MoMLV genome. The resulting elevated gag-pol readthrough had deleterious effects on virus replication.

In the context of the present invention, the term “rpL35” shall include both the mammalian, preferably human, as well as the yeast protein or a functional fragment thereof. While the present invention ultimately aims at pharmaceutical compounds and compositions that are effective in a mammalian, such as a human patient, because of the conservation of the ribosomal proteins, the yeast system has constantly proven to be a valid model for the mammalian situation. Furthermore, the yeast system is more convenient to use as well. The term “rpL35” and/or “functional fragment” shall also include stretches and/or regions of the rpL35 polypeptide that are involved in the modulation of the translation of an mRNA. These areas are involved in binding of the compound (modulator) and/or a subsequent change of the mRNA translation, e.g. by steric hindrance of the mRNA and/or polypeptide as produced by the ribosome. Preferred are functional fragments that include the N-terminal part of rpL35 as disclosed herein, for example in SEQ ID NO: 3, and/or fragments facing the outside of the ribosome (resides on the surface of the ribosome exposed to the surrounding). A general function and functionality for the different mRNAs as disclosed herein (i.e. an mRNA comprising a premature termination codon (PTC), an mRNA that undergoes premature translation termination, programmed −1 ribosomal frameshifting (−1PRF), or the expression of a polycistronic mRNA) can also be assumed because of the exit tunnel position of rpL35 in the ribosome. The fragments can also be used/are useful for binding studies, either for the mRNA to be tested and/or for pre-screening of the modulator.

Preferred is a method according to the present invention, furthermore comprising the step of detecting a binding of said at least one candidate compound to rpL35 or a fragment thereof, preferably to an isolated or partially isolated rpL35 or a fragment thereof, or to rpL35 in the context of the ribosomal subunit or in the context of both subunits of the mammalian ribosome. This aspect relates more to the situation in vivo and in the context of the complete ribosomal structure.

Preferred is also a method according to the present invention, furthermore comprising the step of detecting a binding of said at least one candidate compound to a fragment of rpL35, wherein said fragment comprises from about 70 to about 100 of the N-terminal amino acids of the mammalian rpL35, preferably according to SEQ ID NO: 3. This aspect relates more to the situation in with respect to the positon(s) on rpL35 and the in vitro assays in the context of the present invention.

Further preferred is a method according to the present invention, wherein said detecting of binding (whether in vivo or vitro) comprises detecting an interaction of said at least one candidate compound with an amino acid region of rpL35 selected from the base of helix 2, the loop above helix 3, L9, K13, E15, E67, L69, L95, K97, E99, E100, L102, the set of L9, K13, E15, E67 and L69, and the set of L95, K97, E99, E100 and L102. These sites and regions of rpL35 were identified as being of particular relevance for the binding of compounds, as exemplified for atazanavir and artesunate, see also examples below.

Preferably, detecting of binding to rpL35 or the fragment thereof of the at least one candidate compound(s) is performed as a pre-screening before contacting said at least one candidate compound with said rpL35. That is, the present invention here includes a pre-selection based on the binding properties, e.g. on a recombinant rpL35, before including compounds in the more complex full assays.

Preferred is a method according to the present invention, furthermore comprising a pre-selection step comprising molecular modeling of said binding of said at least one candidate compound to rpL35 or a fragment thereof. This can be done, for example, by using a computer program that identifies candidate compounds by molecular docking and structural analogs thereof, such as SwissDock. That is, the present invention here includes a pre-selection based on the binding properties as modelled in silico, e.g. based on the whole or a part of rpL35, either isolated or in the context of ribosomal proteins, before including compounds in the more complex full in vivo and/or vitro assays.

The above aspect regarding binding can be combined, if desired, e.g. the in silico results can be compared and validated with the in vitro results, and vice versa.

It is assumed in the context of the present invention, that binding of said at least one candidate compound to rpL35 or a fragment thereof constitutes an essential step for the modulation function of said compound. Nevertheless, the assays as described herein also includes pre-testing a binding in the presence or absence of the specific mRNA to be included.

Preferred is the method according to the present invention, wherein said rpL35 or fragment thereof is human rpL35. Comparative analysis of atazanavir binding to yeast and human rpL35 showed that atazanavir binding clusters overlap to some degree, but that on the level of in silico analysis the most prominent group of clusters of atazanavir bound to rpL35 are somewhat distinct for yeast and human (FIG. 3A, B). Despite this, the yeast model system is regarded as sufficiently conserved in order to serve as tool for identifying the situation in human (as exemplified with atazanavir and artesunate herein).

Further preferred is the method according to the present invention, wherein said method is performed in vitro, in cell culture or in vivo, preferably in a non-human mammal. More preferred is the combined assay of in silico binding with human in vitro cell culture assays.

The candidate compound that is to be identified (screened) in the context of the present invention, can be any chemical substance or any mixture thereof. For example, it can be a substance of a peptide library, a library of small organic molecules, a combinatory library, a cell extract, in particular a plant cell extract, a “small molecular drug” (i.e. having a molecular weight of less than about 500 Da), a protein and/or a protein fragment, and an antibody or fragment thereof, and in particular from atazanavir and derivatives thereof and artesunate and derivatives thereof or combinations thereof. Plant extract libraries have proven to be of particular use.

As mentioned above, many orphan diseases are related to the mis-translation of mRNAs based on premature stop codons. Furthermore, many viruses “hijack” the human protein translation machinery (including the ribsosomes) in order to propagate. As therapeutics are missing, the present invention fills in the gap in cases where the at least one mRNA encodes for a protein causing or being associated with Epidermolysis bullosa, viral infections, in particular retroviral infections, such as HIV-1 or coronavirus, like SARS CoV2, such as, for example, LAMB3. It is assumed that the “strategic” position of L35 at the exit tunnel of the ribosome makes it a particularly useful target for the medical approaches as discussed herein.

Another aspect of the present invention then relates to a screening system for identifying a pharmaceutically active compound that modulates the rpL35 (rpL35/rpL29)-dependent translation of at least one mRNA in a mammalian cell, comprising a eukaryotic cell recombinantly expressing a mammalian rpL35 or a fragment of a mammalian rpL35, wherein said fragment comprises from about 70 to about 100 of the N-terminal amino acids of rpL35, an expression construct for recombinantly expressing at least one mRNA to be tested, and optionally, one or more candidate compounds to be tested.

The ribosomal protein rpL35 employed in the methods and systems of the present invention can be a full-length modified protein, or fragments with N/C-terminal and/or internal deletions. Preferably, the fragments are either N-terminal fragments as above. Furthermore, the invention encompasses the use of mutated ribosomal proteins, such as proteins containing amino acid exchanges, modified amino acids, and fusion proteins. Methods for producing mutated ribosomal proteins are well known in the state of the art, and further described herein.

Preferred is a screening system according to the present invention that further comprises a recombinant expression construct, preferably an expression vector, for expressing the mammalian rpL35 or a fragment of a mammalian rpL35, preferably a human rpL35 or fragment thereof, and/or the (in this case proteinaceous or nucleic acid) at least one pharmaceutically active compound to be identified (screened).

Also preferred is a screening system according to the present invention wherein said expression construct for recombinantly expressing at least one mRNA to be tested further comprises at least one suitable reporter group, such as, for example, luciferase reporters. Most preferred is a dual luciferase reporter system, e.g. of luciferases from Photinus pyralis (firefly) and Renilla reniformis.

Basic ribosomal function and structural assembly of its components show a high degree of conservation throughout most biological kingdoms since about 2 billion years of evolution. It is preferred that the eukaryotic cells of the present invention might be selected from a large selection of different eukaryotic model systems, preferably selected from yeast or mammalian cells, such as mouse, rat, hamster (e.g. CHO), monkey or human cells. Not only mammalian cells might be preferred, but also invertebrate cells might be used for such a screening system, including for example insect cells. Preferred is a screening system according to the present invention wherein said eukaryotic cell is selected from a yeast, insect, hamster, or human cell.

Also preferred is a screening system according to the present invention wherein said eukaryotic cell is an inactivation or depletion mutant or comprises other modifications (e.g. posttranslational modifications) of rpL35. The inactivation, depletion or other modifications with respect to ribosomal protein rpL35 shall encompass all alterations (e.g. deletion or mutation) induced with techniques known to the skilled artisan that allow for the functional alteration (inactivation or reduced activity) of a ribosomal protein compared to its wild type state, and/or the alteration of the expression level of said ribosomal protein or its respective mRNA. Enclosed are methods that interfere with appropriate protein function and/or expression at the level of genomic DNA, DNA transcription, mRNA stability and translation, protein expression and post-translational protein trafficking or protein modification. The present invention as an example embodiment thereof uses laboratory strains of cells, wherein the ribosomal protein gene for rpL35 (single and duplicated ribosomal protein genes encode for the 78 or 79 ribosomal proteins in yeast and mammalian cells, respectively) has been inactivated and/or depleted by deletion.

Here, the inventors disclose human ribosomal protein rpL35/uL29 as a target for protein therapy to at least partially overcome and read-through a PTC gene defect in the rare disease Epidermolysis bullosa, ideally leading to the expression of the full-length protein, here LAMB3. In particular, the inventors found that human rpL35/uL29 is able to serve as cellular target for a drug action so that a functional protein is produced from an initial DNA that comprises a nonsense mutation (leading to a premature termination codon, PTC), here in the skin anchor protein LAMB3. The inventors show that in this way a treatment is achieved for the disorder, which is associated with the PTC mutation in the LAMB3 gene.

Similar to the mRNA comprising a premature termination codon (PTC), the present invention can overcome and read-through an mRNA that undergoes premature translation termination, a programmed −1 ribosomal frameshifting (−1PRF), or overcome or “repair” (reduce or inhibit) the expression of a set of proteins derived from a polycistronic mRNA. This will help to treat and alleviate and/or prevent respective disease that are related to these mRNA molecules, such as, for example, Epidermolysis bullosa and other PTC diseases, and viral infections, in particular retroviral infections, such as HIV-1 or HCV or coronavirus, for example SARS CoV2.

Another aspect of the present invention therefore relates to a compound that modulates the rpL35 (rpL35/rpL29)-dependent translation of at least one mRNA in a mammalian cell for use in the prevention or treatment of diseases or condition caused by i) an mRNA comprising a premature termination codon (PTC), ii) an mRNA that undergoes premature translation termination, iii) programmed −1 ribosomal frameshifting (−1PRF), or iv) the expression of a polycistronic mRNA. Preferably, said disease or condition is selected from Epidermolysis bullosa, and viral infections, in particular retroviral infections, such as HCV, HIV-1 or coronavirus, for example SARS CoV2.

Particularly preferred is a compound for use of the invention that modulates the rpL35 (rpL35/rpL29)-dependent translation of at least one mRNA in a mammalian cell which was identified with the enclosed screening systems and/or methods for screening. It is also preferred that the compound according to the present invention can be modified, for example chemically as described further below.

The compound for use and/or that is to be screened in the context of the present invention, can be any chemical substance or any mixture thereof. Preferably, said compound is selected from a chemical substance, a substance selected from a peptide library, a library of small organic molecules (i.e. of a molecular weight of about 500 Da or less), a combinatory library, a cell extract, in particular a plant cell extract, a small molecular drug, a protein and/or a protein fragment, and an antibody or fragment thereof, and in particular from atazanavir and derivatives thereof and artesunate and derivatives thereof.

In the context of the present invention, unless indicated otherwise, the term “about” shall mean+/−10% of the value as given.

The selected or screened compound can then be modified. Said modification can take place in an additional preferred step of the methods of the invention as described herein, wherein, for example, after analyzing the translational activity of rpL35 or the fragment thereof in the presence and absence of said compound as selected, said compound is further chemically modified as described for example, below, and analyzed again for its effect on the translational activity of said ribosomal protein. Said “round of modification(s)” can be performed for one or several times in all the methods, in order to optimize the effect of the compound, for example, in order to improve its specificity for the target protein, and/or in order to improve its specificity for the specific mRNA translation to be influenced. This method is also termed “directed evolution” since it involves a multitude of steps including modification and selection, whereby binding compounds are selected in an “evolutionary” process optimizing its capabilities with respect to a particular property, e.g. its binding activity, its ability to activate, inhibit or modulate the activity, in particular the translational activity of the ribosomal protein rpL35 or the fragment(s) thereof.

The modification can also be simulated in silico before additional tests are performed in order to confirm or validate the effect of the modified selected or screened compound from the first round of screening. Respective software programs are known in the art and readily available for the person of skill.

Modification can further be effected by a variety of methods known in the art, which include without limitation the introduction of novel side chains or the exchange of functional groups like, for example, introduction of halogens, in particular F, Cl or Br, the introduction of lower alkyl groups, preferably having one to five carbon atoms like, for example, methyl, ethyl, n-propyl, isopropyl, n-butyl, isobutyl, tert-butyl, n-pentyl or iso-pentyl groups, lower alkenyl groups, preferably having two to five carbon atoms, lower alkynyl groups, preferably having two to five carbon atoms or through the introduction of, for example, a group selected from the group consisting of NH₂, NO₂, OH, SH, NH, CN, aryl, heteroaryl, COH or COOH group.

Yet another important aspect of the present invention then relates to a method for manufacturing a pharmaceutical composition for the amelioration, prevention or treatment of diseases or condition caused by i) an mRNA comprising a premature termination codon (PTC), ii) an mRNA that undergoes premature translation termination, iii) programmed −1 ribosomal frameshifting (−1PRF), or iv) the expression of a polycistronic mRNA in a subject, comprising the steps of formulating the compound according to the present invention into a suitable pharmaceutical composition, or performing a method according to the present invention for identifying a compound that modulates the rpL35 (rpL35/rpL29)-dependent translation of at least one mRNA in a mammalian cell, and formulating said compound as identified into a suitable pharmaceutical composition. The invention also relates to a pharmaceutical composition obtained by said method according to the present invention. Preferably, said disease or condition is selected from Epidermolysis bullosa, and viral infections, in particular retroviral infections, such as HCV, HIV-1 or coronavirus, for example SARS CoV2, or treating or preventing senescent cells.

Thus, in yet another aspect of the present invention, the selected or screened compound and/or compound for use can be provided and/or is administered as a suitable pharmaceutical composition, such as a topical composition, tablet, capsule, granule, powder, sachet, reconstitutable powder, dry powder inhaler and/or chewable. Such solid formulations may comprise excipients and other ingredients in suitable amounts. Such solid formulations may contain e.g. cellulose, cellulose microcrystalline, polyvidone, in particular FB polyvidone, magnesium stearate and the like. The interacting compound identified as outlined above, which may or may not have gone through additional rounds of modification, is admixed with suitable auxiliary substances and/or additives. Such substances comprise pharmacological acceptable substances, which increase the stability, solubility, biocompatibility, or biological half-life of the interacting compound or comprise substances or materials, which have to be included for certain routes of application like, for example, intravenous solution, sprays, liposomes, ointments, skin crème, band-aids or pills.

It is to be understood that the present compound and/or a pharmaceutical composition comprising the present compound is for use to be administered to a human patient. The term “administering” means administration of a sole therapeutic agent or in combination with another therapeutic agent. It is thus envisaged that the pharmaceutical composition of the present invention are employed in co-therapy approaches, i.e. in co-administration with another, or other medicaments or drugs and/or any other therapeutic agent which might be beneficial in the context of the methods of the present invention. Nevertheless, the other pharmaceutical composition of the present invention, medicaments or drugs and/or any other therapeutic agent can be administered separately from the compound as selected or screened and/or compound for use, if required, as long as they act in combination (i.e. directly and/or indirectly, preferably synergistically) with the present compound as selected or screened and/or for use. See FIG. 8 as an example.

Thus, the compounds as selected or screened and/or for use of the invention can be used alone or in combination with other active compounds—for example with medicaments already known for the treatment of the aforementioned diseases, whereby in the latter case a favorable additive, amplifying or preferably synergistically effect is noticed (see FIG. 8). Suitable amounts to be administered to humans range from 5 to 500 mg, in particular 10 mg to 100 mg. Of course, any dosage can be readily adjusted by the attending physician, if needed, based on, for example, other medical parameters of the patient to be treated.

Pharmaceutical compositions as used may optionally comprise a pharmaceutically acceptable carrier. Pharmaceutically acceptable carriers or excipients include diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, Propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), thickening agents (e.g. methylcellulose or hydroxyethylcellulose), sweetening agents (e.g. sorbitol, saccharin, aspartame, acesulfame), flavoring agents (e.g. peppermint, lemon oils, butterscotch, etc.), humectants (e.g. propylene, glycol, glycerol, sorbitol). Other suitable pharmaceutically acceptable excipients are inter alia described in Remington's Pharmaceutical Sciences, 15^{th} Ed., Mack Publishing Co., New Jersey (1991) and Bauer et al., Pharmazeutische Technologic, 5^{th} Ed., Govi-Verlag Frankfurt (1997). The person skilled in the art knows suitable formulations for respective compounds, for example topical, and will readily be able to choose suitable pharmaceutically acceptable carriers or excipients, depending, e.g., on the formulation and administration route of the pharmaceutical composition.

The therapeutics can be administered orally, e.g. in the form of pills, tablets, coated tablets, sugar coated tablets, hard and soft gelatin capsules, solutions, syrups, emulsions or suspensions or as aerosol mixtures. Administration, however, can also be carried out rectally, e.g. in the form of suppositories, or parenterally, e.g. in the form of injections or infusions, or percutaneously, e.g. in the form of ointments, creams or tinctures.

In addition to the aforementioned compounds as selected or screened and/or for use of the invention, the pharmaceutical composition can contain further customary, usually inert carrier materials or excipients. Thus, the pharmaceutical preparations can also contain additives, such as, for example, fillers, extenders, disintegrants, binders, glidants, wetting agents, stabilizers, emulsifiers, preservatives, sweetening agents, colorants, flavorings or aromatizers, buffer substances, and furthermore solvents or solubilizers or agents for achieving a depot effect, as well as salts for changing the osmotic pressure, coating agents or antioxidants. They can also contain the aforementioned salts of two or more compounds for use of the invention and also other therapeutically active substances as described above.

Yet another aspect of the present invention then relates to a method of modulating the rpL35 (rpL35/rpL29)-dependent translation of at least one mRNA in a mammalian cell, comprising contacting said cell with an effective amount of the compound(s) as selected or screened and/or for use according to the present invention, preferably atazanavir or derivatives thereof and/or artesunate or derivatives thereof. Preferably, said method is a non-medical or cosmetic method, and/or is performed in vivo or in vitro.

Yet another important aspect of the present invention then relates to a method of treating or ameliorating a disease or condition caused by i) an mRNA comprising a premature termination codon (PTC), ii) an mRNA that undergoes premature translation termination, iii) programmed −1 ribosomal frameshifting (−1PRF), or iv) the expression of a polycistronic mRNA in a mammalian cell in a subject in need thereof, comprising administering to said subject an effective amount of the least one compound according to the present invention that modulates the rpL35 (rpL35/rpL29)-dependent translation of said mRNA according to any of i) to iv), or of the pharmaceutical composition according to the present invention, thereby treating or ameliorating a disease or condition

By “treatment” or “treating” is meant any treatment of a disease or disorder, in a mammal, including: preventing or protecting against the disease or disorder, that is, causing, the clinical symptoms of the disease not to develop; inhibiting the disease, that is, arresting or suppressing the development of clinical symptoms; and/or relieving the disease, that is, causing the regression of clinical symptoms. By “amelioration” is meant the prevention, reduction or palliation of a state, or improvement of the state of a subject; the amelioration of a stress is the counteracting of the negative aspects of a stress. Amelioration includes, but does not require complete recovery or complete prevention of a stress.

As above, the compound as administered in the context of the present invention can be any chemical substance or any mixture thereof. Preferably, said compound is selected from a substance selected from a peptide library, a library of small organic molecules (i.e. of a molecular weight of about 500 Da or less), a combinatory library, a cell extract, in particular a plant cell extract, a small molecular drug, a protein and/or a protein fragment, and an antibody or fragment thereof, and in particular from atazanavir and derivatives thereof and artesunate and derivatives thereof.

Preferred is a method according to the present invention, wherein said disease or condition is selected from Epidermolysis bullosa, and viral infections, in particular retroviral infections, such as HCV, HIV-1 or coronavirus, for example SARS CoV2.

In this context it is important to note that in many PTC-associated disease states or conditions a relative small increase in a protein expression from the PTC mRNA (be it by increase in PTC read-through or decrease in the PTC induced decay of the PTC mRNA), is sufficient for a significant improvement of said disease state or condition. In other cases, in turn, it may be desirable to reduce the amount of a target protein, preferably to a minimal level. Examples for this are viral infections.

A rare disease in the United States is defined by the 1983 Orphan Drug Act as a condition that affects fewer than 200,000 people, whereas the analogous definition introduced in the European Union in 2000 considers a disease to be rare when it affects fewer than one in 2,000 people. The currently listed 7000 rare diseases (https://www.eurordis.org) worldwide affect more than 500 Mio people, more than double the number of patients affected by AIDs and cancer combined. Recent advances in annotating mutations in human genes indicate that there may be more than 10,000 rare diseases (https://mondo.monarchinitiative.org/). The majority of rare diseases are genetic disorders, i.e. diseases of Mendelian inheritance.

PTCs account for about 11% of all described gene defects causing human genetic diseases and are often associated with a severe phenotype. The specific genetic mutational event of a PTC mutation during translation of the PTC-affected mRNA leads to premature termination of protein synthesis. A PTC mutation replaces an mRNA sense codon with an unscheduled stop codon/nonsense codon, a signal for termination of protein synthesis. This produces a truncated, potentially non-functional and even harmful protein.

LAMB3635X PTC mutation is the most frequent genetic lesion in rare disease gs-JEB and homozygous loss-of-function variants are postnatal lethal. So far, expansive clinical trials have delivered no approved therapies for these patients. The most advanced clinical trials employ amino glycoside antibiotics. This non-targeted, systemic approach to treat PTC mutations in EB and other rare diseases started several decades ago with the use of aminoglycoside antibiotics for treatment of PTC lesions (Burke J F, Mogg A E. Suppression of a nonsense mutation in mammalian cells in vivo by the aminoglycoside antibiotics G-418 and paromomycin. Nucleic Acids Res. 1985; 13(17):6265-6272). Aminoglycoside antibiotics had been used to treat Gram-negative bacterial infections and in pathogens aminoglycosides bind to a seven-nucleotide loop structure in the decoding center of the bacterial ribosome and decrease the fidelity of decoding mRNA triplets (Fan-Minogue H, Bedwell D M. Eukaryotic ribosomal RNA determinants of aminoglycoside resistance and their role in translational fidelity. Rna. 2008 January; 14(1):148-57). This increases misincorporation of near-cognate tRNAs, resulting in extensive translational misreading of sense codons and stop codons, including premature termination codons. In bacteria, misreading induced by aminoglycosides is neither codon specific nor mRNA specific and results in accumulation of faulty proteins, complete inhibition of protein synthesis and loss of viability of the prokaryotic pathogen. In eukaryotes, a small difference in the rRNA nucleotide sequence of the aminoglycoside binding pocket, adjacent to the decoding center of cytoplasmic ribosomes, significantly lowers the efficiency of drug binding (Lynch S R, Puglisi J D. Structure of a eukaryotic decoding region A-site RNA. J Mol Biol. 2001 Mar. 9; 306(5):1023-35). However, for PTC signals in eukaryotic mRNAs, the impact of aminoglycosides on decoding is often sufficient to reduce fidelity of PTC codon recognition and to promote read-through. In this way, full-length proteins are produced from eukaryotic PTC mRNAs under treatment with aminoglycosides.

Depending on treatment regimens and mode of assessment of production of full-length protein form a PTC mRNA, a series of studies in yeast and human model cells, respectively, report that administration of aminoglycosides increases basal read-through by two to three-fold or generates between 3% to 20% of wild type protein level (reviewed in Dabrowski M, Bukowy-Bieryllo Z, Zietkiewicz E. Advances in therapeutic use of a drug-stimulated translational readthrough of premature termination codons. Mol Med. 2018 May 29; 24(1):25). An increase upon aminoglycoside treatment of 2-fold to 3-fold in production of full-length protein compared to endogenous read-through levels, has been reported to be beneficial in a few studies treating patients with PTC mutations in muscular dystrophy and cystic fibrosis (Allamand V, Bidou L, Arakawa M, Floquet C, Shiozuka M, Paturneau-Jouas M, et al. Drug-induced readthrough of premature stop codons leads to the stabilization of laminin alpha2 chain mRNA in CMD myotubes. J Gene Med. 2008 February; 10(2):217-24, Sloane P A, Rowe S M. Cystic fibrosis transmembrane conductance regulator protein repair as a therapeutic strategy in cystic fibrosis. Curr Opin Pulm Med. 2010 November; 16(6):591-7, Gonzalez-Hilarion S, Beghyn T, Jia J, Debreuck N, Berte G, Mamchaoui K, et al. Rescue of nonsense mutations by amlexanox in human cells. Orphanet Journal of Rare Diseases. 2012 2012/08/31; 7(1):58. At present, the level of PTC read-through for which PTC mRNA has to be achieved to provide reconstitution from the disease phenotype cannot be predicted (Dabrowski et al., 2018). Prolonged application of aminoglycosides, exert strong oto- and nephrotoxic effects on the organism. In addition, aminoglycosides target mitochondrial ribosomes. The induced mistranslation of mitochondrially synthesized proteins of the electron transport chain lead to imbalance of energy metabolism and cause an increased oxidative stress in all cell types of the patient (Kalghatgi S, Spina C S, Costello J C, Liesa M, Morones-Ramirez J R, Slomovic S, et al. Bactericidal antibiotics induce mitochondrial dysfunction and oxidative damage in Mammalian cells. Sci Transl Med. 2013; 5(192):192ra85-92ra85).

To alleviate the complications resulting from aminoglycoside toxicity, various attempts have been undertaken to attenuate aminoglycoside toxicity or to select other compounds with PTC read-through inducing potential, but avoiding undesirable side effects of aminoglycoside antibiotics. These strategies delivered drugs which decrease the activity of the nonsense mediated mRNA decay pathway (Lykke-Andersen S, Jensen T H. Nonsense-mediated mRNA decay: an intricate machinery that shapes transcriptomes. Nat Rev Mol Cell Biol. 2015 November; 16(11):665-77) and thus decrease decay of the PTC mRNA. This provides increased amounts of PTC mRNA to endogenous read-through and delivers increased amounts of full-length protein form the PTC mRNA. Combinatorial use of NMD drugs and read-through drugs has been tried, but with little or no superior effect (Dabrowski et al. 2018, Bukowy-Bieryllo Z, Dabrowski M, Witt M, Zietkiewicz E. Aminoglycoside-stimulated readthrough of premature termination codons in selected genes involved in primary ciliary dyskinesia. RNA Biol. 2016 Oct. 2; 13(10):1041-50). Developing non-aminoglycoside read-through drugs, most notably PTC124 (ataluren), resulted in identification of small molecules with less toxicity, but with no superior performance to aminoglycoside antibiotics, and with no read-through activity for subsets of PTC mRNAs. (Dabrowski et al 2018, Kerem E, Konstan M W, De Boeck K, Accurso F J, Sermet-Gaudelus I, Wilschanski M, et al. Ataluren for the treatment of nonsense-mutation cystic fibrosis: a randomised, double-blind, placebo-controlled phase 3 trial. Lancet Respir Med. 2014 July; 2(7):539-47). A caveat for use of aminoglycosides and other readthrough drugs was reported recently. Studying in mammalian cells the effect of aminoglycoside treatment in a genome wide manner, Wangen and Green (Wangen J R, Green R. Stop codon context influences genome-wide stimulation of termination codon readthrough by aminoglycosides. Elife. 2020 Jan. 23; 9) found unscheduled stimulation of readthrough of a number of PTCs, which are used by the cell as a regulatory mechanism to produce protein isoforms, as well as readthrough of normal termination codons, dysregulating translation of histones and histone modifying enzymes.

In an effort to circumvent the disadvantages of non-discriminatory PTC read-through drugs the inventors thought to develop systemic therapy for all patients carrying the LAMB3635X PTC mutation, in particular for those presenting with homozygosity for this PTC mutation. Translational fidelity is not absolute and in a small percentage of translations reads this allows for basal endogenous readthrough of PTC mRNAs (of 0.1% to 0.01). Early studies have shown that ribosomal proteins can modulate readthrough of PTC mRNAs, but no specificities for distinct PTC mRNAs have been reported. Therefore, the inventors employed the 2SSRS screening tool to identify possible ribosomal protein targets which might serve as ribosomal drug targets specific for systemic repair of LAMB3R635XPTC gene defect (see Bauer et al., 2013 for details).

In this way, the inventors identified ribosomal protein rpL35 as a target ribosomal protein (TRP) for customized increase in full length protein expression of Lamb3R635XPTC, but not that of other PTC mRNAs used as control and with no observable effect on the altered production of mRNAs without PTC mutation (Bauer et al., 2013). The yeast ribosomal protein rpL35 is a structural homologue of the human rpL35 and occupies the identical position on the solvent accessible side of the large 60S ribosomal subunit in yeast and human ribosome, where it adjoins the protein exit tunnel (PET). Therefore the inventors investigated human rpL35 as a possible drug target to customize increase in production of full length Lamb3 protein form the LAMB3R635XPTC mRNA.

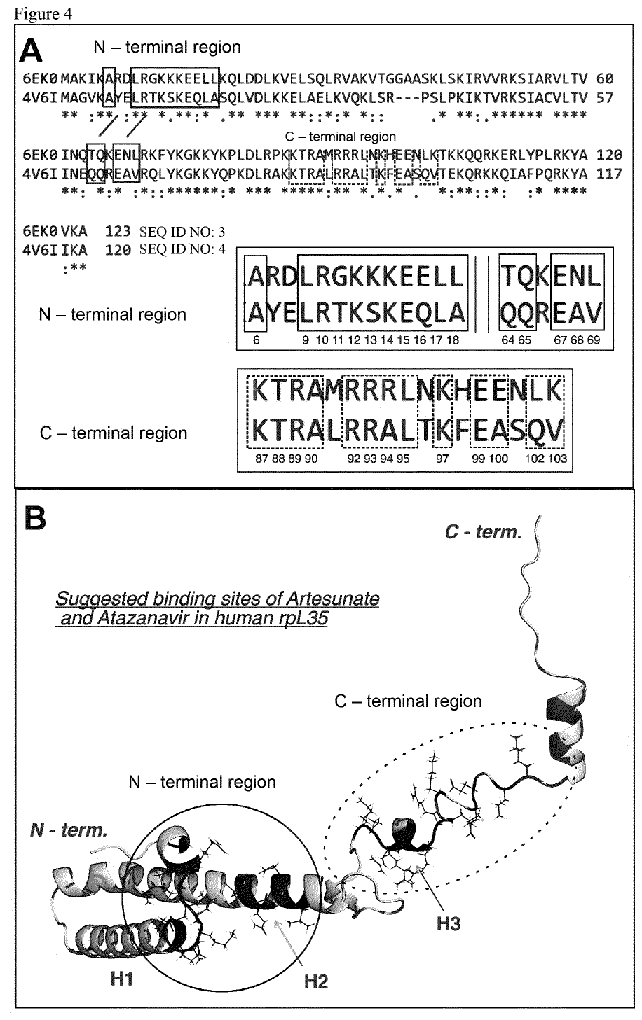

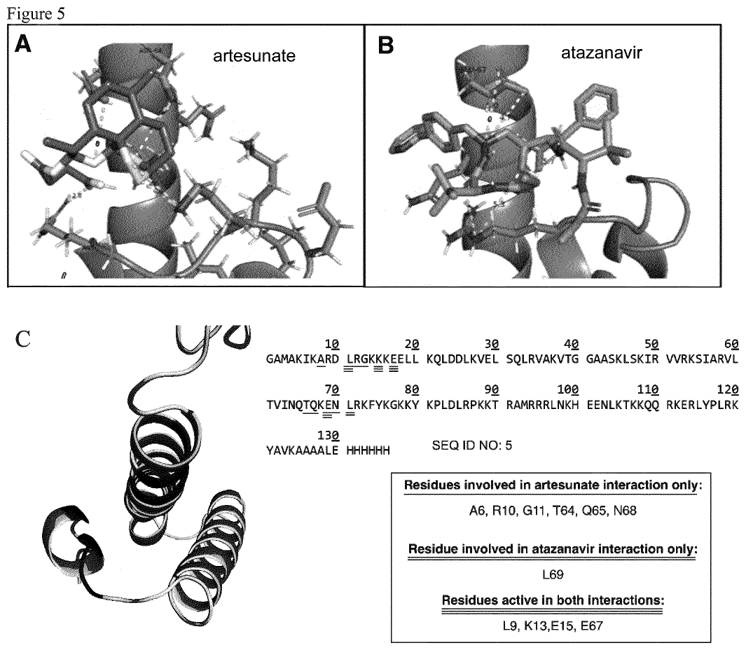

In the context of the present invention, the inventors have identified approved drugs atazanavir and artesunate as candidate small molecule binders of yeast and human rpL35. Repurposable drugs are attractive molecules to test for ligand binding characteristics on target proteins, which like rpL35 have been shown to act as a molecular switch for repair of a disease state. First, the inventors employed molecular docking tools to probe binding sites of atazanavir and artesunate on yeast rpL35 and human rpL35, respectively. The in-silico models indicated overlapping binding clusters of both molecules are scattered along the long axis of yeast rpL35 and human rpL35, with one shared prominent binding cluster targeting the N-terminal site of rpL35, flanked by the rpL35 N-terminal sequence tract. These findings encouraged a more detailed in vitro analysis. Small molecule human rpL35 interaction was assessed by NMR titration series. Analysis of changes in the NMR spectra in the presence of small molecule for both atazanavir and artesunate demonstrated that both RPL35 candidate ligands bind human rpL35 protein at several possible binding pockets, as defined by NMR amino acid shift signals. Such epitope is defined by the amino acid composition providing a complementary electrostatic surface for interaction between small molecules atazanavir and artesunate. The binding epitope was narrowed down to either the N-terminal site, including the N terminal sequence flexible tail, and the more C-terminal site (with small helix 3). Combining the results from NMR titration analysis, bioinformatic docking studies on yeast and human rpL35 and spatial arrangement of rpL35 in the ribosome, the inventors conclude that the most favorable binding site for both Atazanavir and Artesunate is N-terminal site epitope formed by amino acids A6, L9, R10, G11, K13, E15, on the rpL35 N-terminal site and amino acids T64, Q65, E67, N68 and L69 of helix two (FIG. 4B). Although the chemical structures of artesunate and atazanavir are not identical, their overall characteristics (surface charge distribution, shape) are similar. Both have a negatively charged lobe in the center of the molecule and relatively high flexibility in solution which could enable their closer interaction with the protein target. The inventors could discern a drug binding pattern, which targets mostly identical amino acids, although with different coordination parameters. (FIG. 5A-B). Inspection of rpL35 on the ribosome suggests that for rpL35 in its natural state and integrated into the ribosome, the N-terminal site epitope is accessible to both atazanavir and artesunate (see also above for “fragments”).

Here, the inventors have obtained evidence that human ribosomal protein rpL35 binds two established drugs. The first, artesunate, is a member of the artemisinin family. Artemisinin's are herbal compounds serving as anti-malarial agents with a well-established safety profile. The second, atazanavir, is a synthetic tripeptide derivative, used in treatment of HIV infections, however known for a range of side effects. There are several observations that support a customized effect of a rpL35 ligand on tailored increase of LAMB3R635X PTC. First, the ribosome is not a static high molecular complex used for protein synthesis of mRNAs. Rather, subtle changes in composition of the ribosome have been reported to change the overall energy profile, which may impact on the translation rate of only one or a selected set of mRNAs (Choi J, Grosely R, Prabhakar A, Lapointe C P, Wang J, Puglisi J D. How Messenger RNA and Nascent Chain Sequences Regulate Translation Elongation. Annu Rev Biochem. 2018 Jun. 20; 87:421-49. Larsen K P, Choi J, Prabhakar A, Puglisi E V, Puglisi J D. Relating Structure and Dynamics in RNA Biology. Cold Spring Harb Perspect Biol. 2019 Jul. 1; 11(7). Prabhakar A, Puglisi E V, Puglisi J D. Single-Molecule Fluorescence Applied to Translation. Cold Spring Harb Perspect Biol. 2019 Jan. 2; 11(1)). Second, reports are accumulating on ribosomal proteins as modulators of tailored protein production (Shi Z, Fujii K, Kovary K M, Genuth N R, Röst H L, Teruel M N, et al. Heterogeneous Ribosomes Preferentially Translate Distinct Subpools of mRNAs Genome-wide. Mol Cell. 2017 Jul. 6; 67(1):71-83.e7. Sloan K E, Warda A S, Sharma S, Entian K D, Lafontaine D L J, Bohnsack M T. Tuning the ribosome: The influence of rRNA modification on eukaryotic ribosome biogenesis and function. RNA Biol. 2017 Sep. 2; 14(9):1138-52. Genuth N R, Barna M. The Discovery of Ribosome Heterogeneity and Its Implications for Gene Regulation and Organismal Life. Mol Cell. 2018 Aug. 2; 71(3):364-74). Third, the inventors observe that binding of artesunate and atazanavir, respectively, freeze an otherwise more flexible configuration of rpL35 protein. This would support the hypothesis, that binding of these drugs changes in a subtle, but effective way the energy landscape of the translating ribosome to customize increase in basal readthrough rates for LAMB3R635XPTC and thus in production of full length Lamb3 protein. At present, this effect is either by increasing elongation rates for the LAMB3 mRNA, thereby reducing time for recognition of the PTC or by altering rpL35 interaction with the polypeptide exit tunnel, which has been reported to contribute to PTC recognition in bacterial ribosomes or is a complex effect.

The inventors conclude, that the interaction of artesunate and atazanavir with human rpL35 as identified here demonstrates the power of the 2SSRS screen to identify a target ribosomal protein rpL35 and candidate drugs interacting with rpL35 in such a way that customized boost in production of full-length protein from inherited PTC mutants and other mRNA changes as indicated can be achieved. It sets the stage for the functional analysis of these compounds in yeast cells, in human HeLa cell extracts and in general in human cells, designed to carry LAMB3R635XPTC in the homozygous state. In the inventors' case, these experiments show the potential of small molecule rpL35 ligands artesunate and atazanavir, respectively, to act as a molecular switch for rpL35 to customize increased production of full length Lamb3 protein from a LAMB3R635XPTC mRNA.

The present invention preferably relates to the following items
Item 1. A method for identifying a pharmaceutically active compound that modulates the rpL35 (rpL35/rpL29)-dependent translation of at least one mRNA in a mammalian cell, comprising a) contacting rpL35 or a functional fragment thereof with at least one candidate compound in the presence of said at least one mRNA to be translated, and b) detecting the modulation of the translation of said at least one mRNA compared to the translation in the absence of said at least one candidate compound, wherein a modulation of the translation of said at least one mRNA is indicative for said pharmaceutically active compound
Item 2. The method according to Item 1, furthermore comprising a pre-identification of the translation of said at least one mRNA as being rpL35 (rpL35/rpL29)-dependent.
Item 3. The method according to Item 1 or 2, wherein said modulation leads to an increase or decrease of said rpL35 (rpL35/rpL29)-dependent translation of said at least one mRNA.
Item 4. The method according to any one of Items 1 to 3, wherein said at least one mRNA comprises a premature termination codon (PTC), undergoes premature translation termination, causes programmed −1 ribosomal frameshifting (−1PRF), or is a polycistronic mRNA.
Item 5. The method according to any one of Items 1 to 4, furthermore comprising detecting a binding of said at least one candidate compound to rpL35, preferably to an isolated or partially isolated rpL35, or to rpL35 in the context of the ribosomal subunit or in the context of both subunits of the mammalian ribosome.
Item 6. The method according to any one of Items 1 to 4, furthermore comprising detecting a binding of said at least one candidate compound to a fragment of rpL35, wherein said fragment comprises from about 70 to about 100 of the N-terminal amino acids of the mammalian rpL35, preferably according to SEQ ID NO: 3.
Item 7. The method according to any one of Items 1 to 6, wherein said detecting of binding comprises detecting an interaction of said at least one candidate compound with an amino acid region of rpL35 selected from the base of helix 2, the loop above helix 3, L9, K13, E15, E67, L69, L95, K97, E99, E100, L102, the set of L9, K13, E15, E67 and L69, and the set of L95, K97, E99, E100 and L102.
Item 8. The method according to any one of Items 5 to 7, wherein said detecting of binding to rpL35 or the fragment thereof is performed as a pre-screening before contacting said at least one candidate compound with said rpL35.
Item 9. The method according to any one of Items 1 to 8, furthermore comprising a pre-selection step comprising molecular modeling of said binding of said at least one candidate compound to rpL35 or a fragment thereof, for example using a computer program, such as SwissDock.
Item 10. The method according to any one of Items 1 to 9, wherein said rpL35 or fragment thereof is human rpL35.
Item 11. The method according to any one of Items 1 to 10, wherein said method is performed in vitro, in cell culture or in vivo, preferably in a non-human mammal.
Item 12. The method according to any one of Items 1 to 11, wherein said candidate compound is selected from a chemical substance, a substance selected from a peptide library, a library of small organic molecules, a combinatory library, a cell extract, in particular a plant cell extract, a “small molecular drug”, a protein and/or a protein fragment, and an antibody or fragment thereof, and in particular from atazanavir and derivatives thereof and artemisinin and derivatives thereof.
Item 13. The method according to any one of Items 1 to 12, wherein said at least one mRNA encodes for a protein causing or being associated with Epidermolysis bullosa, viral infections, in particular retroviral infections, such as HIV-1 or coronavirus, like SARS CoV2, such as, for example, LAMB3.
Item 14. A screening system for identifying a pharmaceutically active compound that modulates the rpL35 (rpL35/rpL29)-dependent translation of at least one mRNA in a mammalian cell, comprising, a eukaryotic cell recombinantly expressing a mammalian rpL35 or a fragment of a mammalian rpL35, wherein said fragment comprises from about 70 to about 100 of the N-terminal amino acids of rpL35, an expression construct for recombinantly expressing at least one mRNA to be tested, and optionally, one or more candidate compounds to be tested.
Item 15. The screening system according to Item 14, wherein said eukaryotic cell is selected from a yeast, insect, rodent, or human cell.
Item 16. The screening system according to Items 14 or 15, wherein said eukaryotic cell is an inactivation or depletion mutant of rpL35.
Item 17. A compound that modulates the rpL35 (rpL35/rpL29)-dependent translation of at least one mRNA in a mammalian cell for use in the prevention or treatment of diseases or condition caused by i) an mRNA comprising a premature termination codon (PTC), ii) an mRNA that undergoes premature translation termination, iii) programmed −1 ribosomal frameshifting (−1PRF), or iv) the expression of a polycistronic mRNA.
Item 18. The compound for use according to Item 17, wherein said compound is selected from a chemical substance, a substance selected from a peptide library, a library of small organic molecules, a combinatory library, a cell extract, in particular a plant cell extract, a small molecular drug, a protein and/or a protein fragment, and an antibody or fragment thereof, and in particular from atazanavir and derivatives thereof and artenusate and derivatives thereof.
Item 19. The compound for use according to Items 17 or 18, wherein said disease or condition is selected from Epidermolysis bullosa, and viral infections, in particular retroviral infections, such as HIV-1 or coronavirus, for example SARS CoV2.
Item 20. A method of modulating the rpL35 (rpL35/rpL29)-dependent translation of at least one mRNA in a mammalian cell, comprising contacting said cell with an effective amount of atazanavir or derivatives thereof and artenusate or derivatives thereof, or combinations thereof.
Item 21. A method of treating or preventing a disease or condition caused by i) an mRNA comprising a premature termination codon (PTC), ii) an mRNA that undergoes premature translation termination, iii) programmed −1 ribosomal frameshifting (−1PRF), or iv) the expression of a polycistronic mRNA in a mammalian cell, comprising providing an effective amount of at least one compound that modulates the rpL35 (rpL35/rpL29)-dependent translation of said mRNS according to any of i) to iv) to a patient or subject in need of said treatment or prevention.
Item 22. The method according to Item 21, wherein said compound is selected from a chemical substance, a substance selected from a peptide library, a library of small organic molecules, a combinatory library, a cell extract, in particular a plant cell extract, a small molecular drug, a protein and/or a protein fragment, and an antibody or fragment thereof, and in particular from atazanavir and derivatives thereof and artesunate and derivatives thereof.
Item 23. The method according to Item 21 or 22, wherein said disease or condition is selected from Epidermolysis bullosa, and viral infections, in particular retroviral infections, such as HIV-1 or coronavirus, for example SARS CoV2.

The present invention will now be described further in the following examples with reference to the accompanying Figures, nevertheless, without being limited thereto. For the purposes of the present invention, all references as cited herein are incorporated by reference in their entireties.

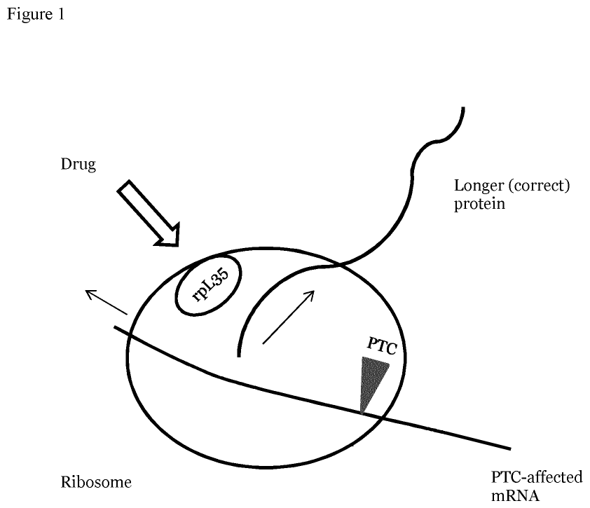

FIG. 1 shows a schematic overview of the approach of the present invention in case of a PTC mRNA.

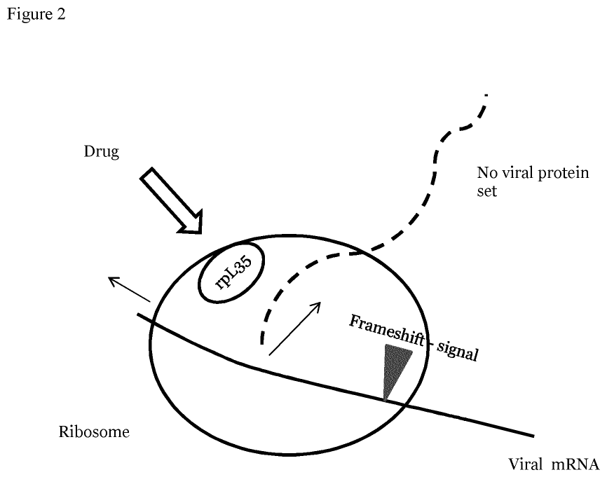

FIG. 2 shows a schematic overview of the approach of the present invention in case of a viral polycistronic mRNA.

FIG. 3 shows the results of blind docking of artesunate and atazanavir to human and yeast orthologs of rpL35. Clusters obtained from SwissDock online server are color coded (red—artesunate; green—atazanavir) and plotted onto single structures of human and yeast rpL35, respectively. Both protein structures were isolated from the cryo-EM structures of the whole ribosomes (Armache J P, Jarasch A, Anger A M, Villa E, Becker T, Bhushan S, et al. Cryo-EM structure and rRNA model of a translating eukaryotic 80S ribosome at 5.5-A resolution. Proc Natl Acad Sci USA. 2010 Nov. 16; 107(46):19748-53; Natchiar S K, Myasnikov A G, Kratzat H, Hazemann I, Klaholz B P. Visualization of chemical modifications in the human 80S ribosome structure. Nature. 2017 Nov. 23; 551(7681):472-77) and the figure was created using PyMOL (The PyMOL Molecular Graphics System, Version 1.2r3pre, Schrödinger, LLC)

FIG. 4 shows the binding domains of human rpL35 for artesunate and atazanavir based on the analysis of NMR titration data. A: Primary sequences of both, human (Natchiar S K, Myasnikov A G, Kratzat H, Hazemann I, Klaholz B P. Visualization of chemical modifications in the human 80S ribosome structure. Nature. 2017 Nov. 23; 551(7681):472-77) (SEQ ID NO: 3) and yeast (Armache J P, Jarasch A, Anger A M, Villa E, Becker T, Bhushan S, et al. Cryo-EM structure and rRNA model of a translating eukaryotic 80S ribosome at 5.5-A resolution. Proc Natl Acad Sci USA. 2010 Nov. 16; 107(46):19748-53), (SEQ ID NO: 4) variants of rpL35. Both potential binding sites are denoted in the sequence, showing also the conserved amino acids. B: Both possibilities for coordination of Artesunate and atazanavir are plotted on the three-dimensional structure of rpL35 isolated from cryo-EM structure of the human ribosome.

FIG. 5 shows coordination of artesunate and atazanavir on the N-terminal domain comprising flexible N-terminus tail, upper part of helix1 and central part of helix2. A: Artesunate docked onto the N-terminal region of rpL35. Distances between functional groups available for electrostatic interactions with oxygen moieties of Artesunate are depicted. B: Results of docking the atazanavir molecule into N-terminal region of rpL35. Possible electrostatic interactions between the ligand and target protein are shown. C: Overlay of binding sites for artesunate and atazanavir, respectively, in the N-terminal region. Amino acids engaged only in the interaction with Artesunate are highlighted in dark gray, the residue that interacts only with atazanavir (L69) is in black and the shared amino acids are in lighter gray.

FIG. 6 shows results of in-vivo dual luciferase reporter read-outs of treated and untreated cells. Luciferase read-out data are represented normalized to the individual luciferase reporter (Ren, Lamb3-FF, Lamb3-PTC-FF) read-outs in the untreated state set at 100% (normalizer); for renilla control—the untreated state—mean and standard deviation of quantifications from twelve biological replicates, each with six technical replicates (72 reads), are shown. For Lamb3-FF control—the untreated state—mean and standard deviation of quantifications from six biological replicates, each with six technical replicates (36 reads), are shown. For Lamb3-PTC control—the untreated state—mean and standard deviation of quantifications from two biological replicates, each with six technical replicates (12 reads), are shown. Quantification upon treatment with ART and ATZ, respectively, for both, Ren (216 reads) and Lamb3-FF (105 reads), is shown by combining read-out data obtained by treatment with 2, 4, and 10 μM ART and ATZ (treated), respectively, and is normalized to the respective untreated control (normalizer). For luciferase reporter read-outs of Lamb3-PTC-FF treated with 2 μM, 4 μM and 10 μM ART and 2, 4, and 10 nM ATZ, respectively, mean and standard deviation of quantification from two biological replicates for ART (12 reads) and three biological replicates for ATZ (18 reads), with six technical replicates each, are shown normalized to Lamb3-PTC-FF control.

FIG. 7 shows the normalized representation of the effect of ribosomal protein rpL35 depletion on increased production of full length Lamb3PTC. Production of dual luciferase reporter proteins pairs REN//Lamb3FF and REN//Lamb3PTC was measured by luciferase assay in extract of Diploid Tetrad Derivatives (DTDs), which were obtained by genetic manipulation in order to generate diploid progeny from diploid parent strains, heterozygous for a depletion in the RPL35 gene (35 A or 35B); each assay was done in 6 technical replicates and in three biological replicates (DTD1, DTD2 and DTD3). Data from the individual reporters, REN, Lamb3-FF and Lamb3PTC-FF were collected. Readout data collected in cells with wildtype genotype were used as normalizers and compared to the readout data obtained in the deletion variants of the DTDs (indicated by Δ). Dotted lines indicate normalization. Unit on the X-axis is normalize to renilla control in percent ([%]). A) DTD1, B) DTD2, C) DTD3.

FIG. 8 shows results of in-vivo production of full-length Lamb3-PTC upon treatment with artesunate, atazanavir, combined artesunate and atazanavir, and erythromycin as dual luciferase reporter read-outs of treated and untreated yeast cells. Luciferase read-out data are represented normalized to the individual luciferase reporter (Lamb3-FF, Lamb3-PTC-FF, FF, and FF-PTC) of a respective control in the untreated state set at 100% (normalizer). Twelve biological replicates (n=12) for all detections. Normalization for treatment is shown, additional controls were run, but are not shown. Dosages: ART 2 μM, ATZ 1.6 nM and erythromycin 4 μM, respectively. Combination treatment ATZ and ART are 50 nM and 0.08 nM, respectively, i.e. 40-fold and 20-fold less than individually applied compounds. The increase of FF-PTC erythromycin is an indicator of the lack of specific activity, compared with ATZ and ART.

### EXAMPLES

No approved targeted systemic therapy is available to PTC mutations in EB, in particular not for gs-JEB. Targeting ribosomal proteins (RP) offers new routes for the treatment of severe inherited diseases such as EB. In yeast and human cells, subpopulations of cytoplasmic ribosomes can be generated, by providing altered functional availability of individual ribosomal proteins. Such heterologous or specialized ribosomes are tailored to increase or decrease protein expression of selected mRNAs, while leaving bulk protein expression unaltered. The present invention explores that small molecules binding to rpL35 can be found that offer new routes for the treatment of severe inherited diseases, such as EB.

In the present examples, therefore, binding and nature of the interaction of a small molecule with the rpL35 protein is analyzed by titration as monitored by specific interaction by NMR spectroscopy in solution. This provides a proof of concept for drug development of small molecules binding to target ribosomal protein rpL35, as exemplary identified as a ribosomal switch to increase protein production of full length Lamb3PTC protein in gs-JEB.

Materials and Methods

Molecular Docking Studies

The structure of rpL35 was separated from complex cryo-EM structure of the human ribosome (Natchiar S K, Myasnikov A G, Kratzat H, Hazemann I, Klaholz B P. Visualization of chemical modifications in the human 80S ribosome structure. Nature. 2017 Nov. 23; 551(7681):472-77.), and the rpL35 PDB file was loaded into Swiss Dock (SwissDock database (http://www.swissdock.ch/)). Electrostatic surface of rpL35 protein was determined by assessment of the UCSF Chimera software (Petersen et al., J Comput Chem, 2004) adjusted for pH and charge of the respective amino acid side chains by using the Adaptive Poisson-Boltzmann Solver (APBS) function. Subsequently docking studies were performed (SwissDock) by scanning the structure for small molecule binders yielding the best hit for affinity kinetics based on the ΔG values and on available structures from the Swissdock database.

The best hits were isolated and checked for structural analogues in order to arrive at practically accessible molecule that the inventors would be able to test in in vitro binding studies. The most promising rpL35 binding candidate was CPG53820, an immediate precursor of Atazanavir, a commercially available drug, which was employed for further analysis. A screening biotinylation assay delivered Artesunate as a binder of human rpL35 (Ravindra K C, Ho W E, Cheng C, Godoy L C, Wishnok J S, Ong C N, et al. Untargeted Proteomics and Systems-Based Mechanistic Investigation of Artesunate in Human Bronchial Epithelial Cells. Chem Res Toxicol. 2015 Oct. 19; 28(10):1903-13). In an analogous fashion to the Atazanavir docking, the inventors probed binding of Artesunate to human and yeast rpL35 proteins. This in silico analysis encouraged studies to investigate binding of the small molecules to rpL35 in solution, which approaches in vivo situation. The inventors opted for protein solution NMR spectroscopy, as this reflects dynamics of ligand binding and informs on rpL35 residues participating in ligand interaction.

Molecular Cloning

The open reading frame coding for C-terminal His₆ tagged human rpL35 (NCBI ID: 11224) was PCR amplified from a verified vector using the primers F-5′CATGCCATGGC-CAAGATCAAGGCTC′3 (SEQ ID NO: 1) and R-5′CTCTAGATTCAGTCAGATCTCAGTG′3 (SEQ ID NO: 2) containing the restriction consensus sequences for NcoI and XbaI, respectively (PCR conditions: 95° C. —5 min, 42×[94° C. —′30, 61° C. —′30, 72° C.—′30], 72° C.—10 min). The resulting amplicon was ligated into the restriction linearized pMBP-parallel 1 expression vector destined for recombinant protein expression in E. coli. The engineered expression plasmid contains an MBP-rpL35-His₆ double tag construct with a TEV cleavage site between MBP and rpL35. Recombinant protein expression is under the control of an IPTG inducible T7 promoter and ampicillin selection marker. Vector sequences were controlled by sequencing construct.

Transformation and Expression

The transformation of the expression plasmid into the E. coli competent cells was performed with the heat shock method. Briefly, 50 μL of log-phase chemically competent E. coli BL21 cells (NEB C2523H) were mixed with 1 μL (1 mg/μL DNA) of the corresponding expression vector, carrying the human rpl35 sequence with N-terminal MBP tag and C-terminal His₆ tag, incubated for 10 min on ice followed by a 30 sec heat shock at 42° C. and 10 min resting on ice. To the transformation mix 500 μL of Luria-Bertani (LB) medium was added, and the sample was pre-cultivated for 1 h at 37° C. Then 100 μL of the transformation mix were plated on agar plates, supplemented with the selection marker ampicillin (100 μg/mL) and incubated overnight at 37° C. Single colony was picked and transferred into 20 mL of LB medium and cultivated for 4 h at 37° C., 180 rpm. 2.5 mL of this culture was transferred into 250 mL of LB medium and cultivated at 37° C. (150 rpm) until optical density (OD₆₀₀) reached 0.6. Culture was centrifuged for 15 min at 25° C. (1500 g) and the pellet was gently resuspended in 250 mL of ¹⁵N isotopically labeled minimal medium M9, pH-optimized (Cai et al., J Biomol NMR, 2016). The cells were then cultivated at 37° C. (150 rpm) for approx. 40 min until OD₆₀₀ reached 0.8. Temperature was lowered to 28° C. and protein expression was induced with IPTG (final conc. 1 mM). The cells were cultivated for 18 hours, at 150 rpm, using the minimal time for ensuring sufficient expression of native aggregation-prone protein, avoiding accumulation of aggregates in inclusion bodies. The bacterial culture was centrifuged (4° C., 4700 g, 1 h), and the cell pellets were stored at −20° C. overnight.

Protein Purification

The cell pellets were resuspended in 10 mL of 50 mM Na₂HPO₄, 300 mM NaCl, 0.1% Triton, pH 7.4 and sonicated at 10% amplitude (maximum power 10 W, Fisher Scientific™Model 705). After centrifugation (27000 g, 4° C., 20 min) the lysate was slowly loaded on 5 mL MBP column (MBPTrap™ HP, GE Healthcare). Upon column equilibration, the MBP-rpL35-His₆ fusion protein was eluted using elution buffer gradient from 0 to 50%, supplemented with 10 mM maltose competitor. The purity and relative concentration was estimated by SDS-PAGE (not shown). The eluate was brought into 20 mM Tris, 150 mM NaCl, 5% glycerol, 5 mM β-mercaptoethanol, 0.5 mM EDTA, pH 7.4 using Amicon® ultra-centrifugal filters (Merck) with 10 kDa cutoff and concentrated to 3 ml. TEV protease (2.3 mg/mL) was added at a volume ratio of 1:100, and the reaction mixture was incubated at room temperature for 3 hours, a condition found to yield maximum intact protein. However, as the majority of the cleaved rpl35-His₆ protein was poorly soluble, chaotropic buffer (20 mM Na₂HPO₄, 8 M urea, 10 mM imidazole, 500 mM NaCl, pH 7.4) was used to completely dissolve the protein sample. This soluble fraction was loaded on a HisTrap column (GE Healthcare), equilibrated with the running buffer, and urea was completely eliminated by addition of refolding buffer. The protein was eluted with 500 mM imidazole gradient, and protein purity was confirmed by SDS-PAGE. Prior to NMR measurements, the rpL35-His₆ protein sample was brought into 20 mM Bis-Tris, 300 mM NaCl, 150 mM glycine, protease inhibitors, 10% D20, pH 6.0 using Amicon® ultra-centrifugal filters (Merck) with 3 kDa cutoff and concentrated to 350 μL. The protein concentration was determined by UV-Vis spectroscopy at 280 nm (Shimadzu 1800).

NMR Spectroscopy

NMR experiments were recorded at 25° C. (298K) on a 700 MHz Bruker Ascend spectrometer equipped with cryogenically cooled TCI probe. All spectra were processed using Topspin 3.5 and analyzed with CARA 1.8.4.2. (Keller R. Optimizing the Process of Nuclear Magnetic Resonance Spectrum Analysis and Computer Aided Resonance Assignment. ETH Zurich; 2004) and NMRFAM-Sparky (Lee W, Tonelli M, Markley J L. NMRFAM-SPARKY: enhanced software for biomolecular NMR spectroscopy. Bioinformatics. 2015 Apr. 15; 31(8):1325-7). Concentration of ¹⁵N labeled rpL35-His₆ ranged from 200 to 400 μM and the sample was measured in a salt tolerant susceptibility matched slot tube (Shigemi™) with total volume of 170 μl.

NMR spectroscopy is able by comparative analysis of protein vs. protein+small molecule binder to asses binding sites of the small molecule on the protein. In this initial study the affinity of rpL35 towards artesunate and atazanavir was established by a simple titration series in which the inventors have been tracking perturbations to chemical shifts and peak heights in the 2D ¹H, ¹⁵N HSQC spectrum. Aliquots of artesunate/atazanavir were added into the solution of ¹⁵N rpL35 of which 2D 1H, ¹⁵N HSQC spectrum was recorded immediately upon ligand addition. Perturbations of protein chemical shifts in ¹⁵N HSQC spectrum are indicative of changes to the chemical environment of amide groups caused by binding of a ligand to the protein target.

Following the artesunate-protein rpL35 complex titration, the inventors have recorded 3D spectra of the same sample using ¹⁵N NOESY-HSQC, ¹⁵N TOCSY-HSQC, HNHA pulse sequences. The inventors extracted chemical shifts of amide ¹H, ¹⁵N, HA, HB from the above mentioned spectra, and compared measured values with the average chemical shifts of common amino acids in Biological Magnetic resonance Data Bank (BMRB; http://www.bmrb.wisc.edu). In the 3D ¹H, ¹⁵N NOESY-HSQC the inventors focused also on the presence of water signal which can indicate if the amino group is solvent accessible. At this point of the study the inventors did not pursue full resonance assignment, usually based on a set of 3D triple resonance spectra (¹H, ¹⁵N, ¹³C) as this requires doubly isotopicaly labeled protein (¹⁵N, ¹³C). Moreover, nature of rpL35 which possesses a large degree of intrinsically disordered regions hinders acquisition of high-resolution 3D triple resonance experiments to some extent as most of the signals have a low dispersion in proton dimension of the spectra and many signals are overlaid. However, collection of the basic set of chemical shifts mentioned earlier allowed the inventors to estimate amino acid type since many of them have quite characteristic values (e. g. alanine, threonine, and glycine). 3D ¹⁵N TOCSY-HSQC helped the inventors to differentiate between amino acid types which have similar NH chemical shifts, such as leucine, lysine, valine. This spectrum shows for each amino group (one amino acid) all proton signals coupled to a carbon atom, i.e. protons coming from an aliphatic side chain. Using this approach the inventors obtained a set of protein signals which changed their position in 2D ¹H¹⁵N-HSQC spectrum upon interaction with the ligand (artesunate, atazanavir) and information on the most probable amino acid type. The inventors mapped this set of prospective amino acids on the tertiary structure of human rpL35 and thus identified a “hotspot” region where artesunate and atazanavir, respectively, are biding. PDB files of artesunate and atazanavir were first minimized in water using YASARA software and then docked onto selected regions of human rpL35 based on the results of NMR titration using AutoDock Vina feature of the Chimera software (Trott O, Olson A J. AutoDock Vina: improving the speed and accuracy of docking with a new scoring function, efficient optimization, and multithreading. Journal of computational chemistry. 2010; 31(2):455-61).

Bioinformatic Docking Studies Show Binding of Atazanavir to Yeast and Human Ribosomal Protein RPL35

It was found in the context of the present invention that for a development of systemic therapy in PTC disease, such as EB, the human ribosomal protein RPL35 will have to be targeted. As the yeast and human ribosomal protein are very similar on sequence level, secondary and tertiary structure, and the spatial arrangement on the ribosome, the inventors opted to perform a bioinformatic docking analysis. Human rpL35 was used as isolated form the cryo-EM structure of complete ribosome (Natchiar S K, Myasnikov A G, Kratzat H, Hazemann I, Klaholz B P. Visualization of chemical modifications in the human 80S ribosome structure. Nature. 2017 Nov. 23; 551(7681):472-77), and as the most promising candidate ligand compound CPG53820 was identified, showing optimal binding kinetics and a ΔG value of −8.3 kcal/mol. The ΔG value indicates protein-ligand binding characteristics in the range of optimal binding capacities (Du, et al., 2016). CPG53820 is an immediate precursor of the compound atazanavir, an FDA approved drug, and therefore further studies were performed using this molecule.

Docking of atazanavir to the surface of rpL35 then showed several clusters of possible ligand coordination, scattered along the complete length of the protein (FIG. 3A), with one prominent cluster located at the base of the longer of the two major alpha helices, helix 2. These results suggested to investigate potential binding sites of atazanavir on yeast rpL35 that was identified in the yeast screening tool 2SSRS as a prospective target ribosomal protein for customized increase in Lamb3PTC protein expression (Bauer et al., 2013). Again, binding of atazanavir was observed on multiple regions of yeast rpL35 protein, however the biggest cluster resides in the central sequence tract of helix 2, and extends to N-terminal region, which adjoins helix 2 (FIG. 3B).

Comparative analysis of atazanavir binding to yeast and human rpL35 then showed that atazanavir binding clusters overlap to some degree, but that on the level of in silico analysis the most prominent group of clusters of atazanavir bound to rpL35 are somewhat distinct for yeast and human (FIG. 3A, B).

Molecular Interactions Analysis Shows Binding of Artesunate to Yeast and Human Ribosomal Protein RPL35

A biotinylation assay identified artesunate, an FDA approved drug, also as a binder to human rpL35 (Ravindra, K. C., 2015). In analogous fashion to the atazanavir docking studies, the inventors probed binding of artesunate to human and yeast rpL35 proteins.

For human rpL35, the inventors observed multiple clusters of artesunate binding, with the most prominent one residing in the central region of helix 2 (FIG. 3A). For the yeast variant, several clusters were detected, with the largest cluster overlapping the atazanavir helix 2 and N-terminus central binding domain (FIG. 3B).

When the respective binding clusters of atazanavir and artesunate on yeast and human rpL35 are compared, the most distinct binding cluster of artesunate for both, yeast and human rpL35, maps to the central domain of helix 2 and flexible N-terminal sequence tract (N-terminal site). For atazanavir, the most prominent binding cluster in human rpL35 is at the base of helix 2, whereas in yeast, the majority of bound molecules are located at the N-terminal site, overlapping with the respective binding sites of artesunate in both proteins. In addition, there are minor sets of bound atazanavir and artesunate which overlap in both yeast and human rpL35 close to C-terminal region of the protein (helix 3; see FIG. 3A, B).

This initial molecular interactions analysis by Swissdock encouraged studies to investigate binding of candidate small molecule binders to human rpL35 in solution, which reflects a more in vivo situation. The inventors opted for protein solution NMR spectroscopy, as this reflects dynamics of ligand binding and informs on rpL35 amino acid participating in ligand interaction.

NMR Spectroscopy

Initial inspection of the protein by 2D ¹H, ¹⁵N HSQC (Heteronuclear Single Quantum Coherence) spectrum revealed that rpL35-His₆ is prone to form soluble aggregates, which the inventors found to be dissolved by addition of detergent, sodium dodecyl sulphate (at 0.5% concentration). Since the presence of detergent could mask possible molecular interaction between protein and ligand, the inventors have optimized the preparation protocol to arrive at substituting SDS with a buffer of high ionic strength (300 mM NaCl) as well as glycine (150 mM). Enrichment of this buffer with protease inhibitors (cOmplete™ EDTA-free protease inhibitor cocktail, Roche, used according to the manufacturer's instructions) prevented aggregation and precipitation. This set the stage for long-term NMR experiments.

In a first approach, the 2D ¹H, ¹⁵N HSQC spectrum revealed 95 resolved cross peak signals which corresponds to roughly 70% of 136 amino acids of the rpL35-His₆ sequence. This lower figure reflects both the nature of the protein fold, which is a partially disordered one, as well as the amino acid composition of rpL35, which harbors a substantial number of similar amino acids (26 lysines, 17 leucines, 15 arginines, 13 alanines). The intrinsically disordered nature of the rpL35 protein becomes evident by the presence of a significant peak overlap in the central region of the spectrum and the overall rather narrow spectral width in the proton dimension of the spectrum. Aggregate formation of rpL35 was excluded by the observation that comparative analysis of the main HSQC spectrum and its TROSY derivative did not reveal any additional peaks, excluding formation of a high-molecular weight species.

¹H ¹⁵N NOESY-HSQC spectrum allowed to estimate which amide pairs are more water-accessible. Approximately 20% of the peaks did not have a NOE signals coupling to water in their vicinity, suggesting that they are more buried in the protein structure. This would correspond to residue on the interfaces between helices 1 and 2. Combining results from various NMR experiments, the inventors conclude that rpL35 in the optimized buffer is a monomeric molecule that is highly dynamic in solution, yet possesses a stable conformation, compatible with rpL35 three-dimensional structure as reported from cryo-EM analysis (Natchiar S K, Myasnikov A G, Kratzat H, Hazemann I, Klaholz B P. Visualization of chemical modifications in the human 80S ribosome structure. Nature. 2017 Nov. 23; 551(7681):472-77).

NMR Spectroscopy Reveals Two Candidate Regions of rpL35 for Interaction with Artesunate

Chemical shift perturbations observed for a specific group of signals upon the addition of artesunate are of similar magnitude and have rather linear trend. These residues are likely engaged in the same interaction, i.e. binding of the artesunate molecule. Based on the chemical shifts of amide ¹H, ¹⁵N, HA and HB of the perturbed peaks the inventors have mapped residues (FIG. 4A) onto 3D structure of rpL35 from the cryo-EM structure of the ribosome (PDB: 6EK0 Natchiar S K, Myasnikov A G, Kratzat H, Hazemann I, Klaholz B P. Visualization of chemical modifications in the human 80S ribosome structure. Nature. 2017 Nov. 23; 551(7681):472-77) and identified two alternative candidate binding sites (FIG. 4B). The first one is at the intersection of helix1, helix2 and the flexible N-terminus tail, which forms a small cleft. This N-terminal region comprises of charged amino acids such as glutamate, arginine and lysine accompanied by hydrophobic residues including leucine, alanine and glycine. In total, the inventors see clear perturbations to 10 protein signals upon artesunate addition. On the N-terminal region of human rpL35, these amino acids correspond to the inventors' experimental data: A6, one of L9/L17/L18, R10, G11, one of K12/K13/K14, E15 or E16, T64, Q65, E67 and N68. The second location that could accommodate such combination of amino acids is closer to the C-terminus of rpL35, formed by helix3 and a flexible loop in the C-terminal region. In this case, the following residues engage in the interaction between human rpL35 and artesunate: G75, K87 or K97, T88, A90, two out of R92/R93/R94/R89, E99, E100, L95 or L102.

The position of rpL35 in the ribosome favors the first binding site closer to N-terminus as this whole protein region forms a cleft freely accessible from the solvent side. C-terminal region is more tangled below the RNA and makes this sterically less available for any interaction with small organic molecule (see FIG. 5C). Moreover, rpL35 is a dynamic protein and contains a high degree of disorder that is accumulated mostly in the C-terminal region. Such characteristics make this available site rather unfavorable for binding small molecule, unless the binding of artesunate induces change in the secondary and possibly tertiary structure. However, such shift in the conformation would correspond to more pronounced changes to the spectrum which the inventors did not observe. Swissdock prediction of molecular interactions between artesunate and both, human and yeast rpL35 showed most of binding sites clustered in an analogous site to the N-terminal region defined with the inventors' experiments (see FIG. 3A). Result of the docking of artesunate to N-terminal region of human rpL35 using Chimera software allowed the inventors to narrow down the most probable residues engaged in the interaction (FIG. 5A): A6, R10, G11, K13, E15, L17, T64, Q65, E67 and N68. Surprisingly, most of these amino acids are conserved among the human and yeast protein (FIG. 4A). The inventors suggest that these rpL35 residues interact with artesunate in solution.

Atazanavir Shares Binding Site with Artesunate

NMR titration of human rpL35 with atazanavir in solution triggered similar changes to the HSQC spectrum. In the case of atazanavir, only 5 amide groups exhibited significant chemical shift perturbations compared to 10 perturbed signals in the artesunate titration. Yet all except one were also affected in the artesunate titration. One of the peaks, #78 which corresponds to alanine (assumed to be A6, based on the mapping of perturbed signals to protein structure) experienced changes to its chemical shift in the artesunate titration, but only showed decrease in its intensity in the atazanavir experiment. This can be explained by two different effects being caused by addition of either of the ligands. Change in the chemical shift of a peak corresponds to the change in its chemical environment caused by the interaction (electrostatic interactions, hydrogen bonding) with the drug. Drop in the intensity of a peak is a result of a change in the dynamic behavior of this atom, its chemical exchange rate with the solvent may be altered, e.g. the aliphatic bulk of the ligand might shield the amide hydrogen. This suggests that artesunate and atazanavir share the same binding pocket, but that the role of individual amino acids engaged in the interactions differs slightly. Mapping the candidate residues on N-terminal region the following residues were found: one of L9/L17/L18, one of K12/K13/K14, E15 or E16, E67 and L69. Out of these, the inventors suggest that L9, K13, E15, E67 and L69 create the most eligible pocket to accommodate for atazanavir. As the similarity of the spectra indicates that this binding site is near identical for artesunate and atazanavir, the inventors searched for available residues in the C-terminal region that could also correspond to the NMR chemical shift perturbation data. The set of L95, K97, E99, E100 and L102 is located above helix3, in the unstructured loop of rpL35.

Prediction of atazanavir binding sites to human and yeast rpL35 by SwissDock was not conclusive as the highest number of bound ligand molecules was located in different regions of the two proteins (base of helix2 in human and in the N-terminal central region in yeast; FIG. 3A-B). When the inventors take into account the fact that for artesunate most of the ligand is situated in one site both in human and yeast rpL35 (here denoted as the N-terminal region) and that the inventors have strong experimental evidence that the binding sites of artesunate and atazanavir overlap, the inventors conclude that the N-terminal region is the more plausible interaction site of rpL35 with both drugs. This site is apparently more freely accessible for any ligand as it resides on the surface of the ribosome and is exposed to the solvent (FIG. 5C).

Artesunate and Atazanavir in Functional Yeast Assays Customize Increase in Production of Full Length LAMB3PTC

Obtaining evidence for drug directed manipulation of rpL35 to selectively increase full-length Lamb3-PTC expression has to be tested in functional assays. The inventors opted to first test small molecule action in the versatile yeast cell factory, employing the dual luciferase reporter system used in the original 2-SSRS (Bauer et al., 2013).

As shown in FIG. 6, there is no significant increase in protein expression level upon small molecule treatment for the renilla luciferase reporter and the Lamb3-FF luciferase reporter, respectively. This is the case both, for the treatment with ART and ATZ. However, there is a highly significant increase (2.2-fold) in full-length Lamb3-PTC-FF protein expression upon treatment with 2 μM ART. Minimal, not significant increase for Lamb3-FF protein expression was observed upon treatment with 4 μM ART (1,1-fold) and 10 μM ART (1.2-fold). There is a highly significant increase in full-length Lamb3-PTC-FF protein expression upon treatment with 2 μM ATZ (1.8-fold), a similar but not significant increase upon treatment with 4 μM ATZ (1.9-fold) and a significant increase upon treatment with 10 μM ATZ (2.2-fold).

The inventors conclude that rpL35 ligands ART and ATZ trigger increase in full-length protein expression of Lamb3-PTC-FF but not of Lamb3-FF nor of Ren. The fold increase in full-length Lamb3-PTC-FF expression is in the range reported for change in protein expression levels triggered by modulation of ribosomal proteins, i.e. two-fold up and two-fold down, compared to the unaltered state. In summary, these experiments confirm that treatment with small molecules, which act as putative modulators of a distinct ribosomal protein customize increase in protein expression of a selected mRNA species, specifically controlled by that ribosomal protein. Furthermore, these experiments confirm that small molecule treatment, aiming to modulate a distinct ribosomal protein, is a selective treatment in that it tailors the expression levels of a target protein, but not of control proteins.

Preliminary results also showed expression of LAMB3 Expression in human cells as treated using a highly sensitive instrument (Bruker TimsTOF Plus), as the reverted expression according to the present invention often is still much lower (e.g. 100 times) lower than in the wild type.

Ribosomal Protein rpL35 is a Robust Target for Screening

FIG. 7 shows that depletion of rpL35A and depletion of rpL35B do not alter protein expression level of REN and Lamb3-FF luciferase reporters. However, for both the rpL35A depletion and the rpL35B depletion the inventors observed a significant increase in protein expression levels of Lamb3PTC-FF reporter. The analysis using normalized representation of the deletion phenotypes shows that ribosomal protein rpL35, encoded by either paralog, rpL35A or rpL35B, is a robust target for mediating customized increase in production of full length Lamb3PPTC protein. In sum this study supports the hypothesis that ribosomal protein rpL35 is a robust target ribosomal protein for the customized increased production of full length Lamb3 PTC protein.

1. A method for identifying a pharmaceutically active compound that modulates the rpL35 (rpL35/rpL29)-dependent translation of at least one mRNA in a mammalian cell, comprising
a) contacting rpL35 or a functional fragment thereof with at least one candidate compound in the presence of said at least one mRNA to be translated, and
b) detecting the modulation of the translation of said at least one mRNA compared to the translation in the absence of said at least one candidate compound, wherein a modulation of the translation of said at least one mRNA is indicative for said pharmaceutically active compound.

2. The method according to claim 1, furthermore comprising a pre-identification of the translation of said at least one mRNA as being rpL35 (rpL35/rpL29)-dependent.

3. The method according to claim 1, wherein said modulation leads to an increase or decrease of said rpL35 (rpL35/rpL29)-dependent translation of said at least one mRNA.

4. The method according to claim 1, wherein said at least one mRNA comprises a premature termination codon (PTC), undergoes premature translation termination, causes programmed −1 ribosomal frameshifting (−1PRF), or is a polycistronic mRNA.

5. The method according to claim 1, furthermore comprising detecting a binding of said at least one candidate compound to rpL35.

6. The method according to claim 1, furthermore comprising detecting a binding of said at least one candidate compound to a fragment of rpL35, wherein said fragment comprises from about 70 to about 100 of the N-terminal amino acids of the mammalian rpL35.

7. The method according to claim 1, wherein said detecting of binding comprises detecting an interaction of said at least one candidate compound with an amino acid region of rpL35 selected from the base of helix 2, the loop above helix 3, L9, K13, E15, E67, L69, L95, K97, E99, E100, L102, the set of L9, K13, E15, E67 and L69, and the set of L95, K97, E99, E100 and L102.

8. The method according to claim 5, wherein said detecting of binding to rpL35 or the fragment thereof is performed as a pre-screening before contacting said at least one candidate compound with said rpL35.

9. The method according to claim 1, furthermore comprising a pre-selection step comprising molecular modeling of said binding of said at least one candidate compound to rpL35 or a fragment thereof.

10. The method according to claim 1, wherein said rpL35 or fragment thereof is human rpL35.

11. The method according to claim 1, wherein said method is performed in vitro, in cell culture or in vivo.

12. The method according to claim 1, wherein said candidate compound is selected from a chemical substance, a substance selected from a peptide library, a library of small organic molecules, a combinatory library, a cell extract, and an antibody or fragment thereof.

13. The method according to claim 1, wherein said at least one mRNA encodes for a protein causing or being associated with Epidermolysis bullosa or a viral infection.

14. A screening system for identifying a pharmaceutically active compound that modulates the rpL35 (rpL35/rpL29)-dependent translation of at least one mRNA in a mammalian cell, comprising:
a eukaryotic cell recombinantly expressing a mammalian rpL35 or a fragment of a mammalian rpL35, wherein said fragment comprises from about 70 to about 100 of the N-terminal amino acids of rpL35, and
an expression construct for recombinantly expressing at least one mRNA to be tested, and
optionally, one or more candidate compounds to be tested.

15. The screening system according to claim 14, wherein said eukaryotic cell is selected from a yeast, insect, rodent, or human cell.

16. The screening system according to claim 14 or 15, wherein said eukaryotic cell is an inactivation or depletion mutant of rpL35.

17-18. (canceled)

19. A method of modulating the rpL35 (rpL35/rpL29)-dependent translation of at least one mRNA in a mammalian cell, comprising contacting said cell with an effective amount of atazanavir or derivatives thereof and artesunate or derivatives thereof or combinations thereof.

20. A method of treating or ameliorating a disease or condition caused by i) an mRNA comprising a premature termination codon (PTC), ii) an mRNA that undergoes premature translation termination, iii) programmed −1 ribosomal frameshifting (−1PRF), or iv) the expression of a polycistronic mRNA in a mammalian cell, comprising providing an effective amount of at least one compound that modulates the rpL35 (rpL35/rpL29)-dependent translation of said mRNA according to any of i) to iv) to a patient or subject in need of said treatment or prevention.
